(19) United States
(12) Patent Application Publication
(10) Pub. No.: US 2016/0024585 A1
(43) Pub. Date: Jan. 28, 2016

(54) METHODS OF PREDICTING RESPONSIVENESS OF A CANCER TO AN AGENT AND METHODS OF DETERMINING A PROGNOSIS FOR A CANCER PATIENT

(71) Applicant: DUKE UNIVERSITY, Durham, NC (US)

(72) Inventors: Andrew B. Nixon, Durham, NC (US); Herbert I. Hurwitz, Durham, NC (US); Stephanie Mackey Cushman, Durham, NC (US); Chen Jiang, Durham, NC (US); Ivo Shterev, Durham, NC (US); Kouros Owzar, Durham, NC (US); Ace J. Hatch, Durham, NC (US); Alexander B. Sibley, Durham, NC (US)

(73) Assignee: DUKE UNIVERSITY, Durham, NC (US)

(21) Appl. No.: 14/703,535

(22) Filed: May 4, 2015

Related U.S. Application Data
(60) Provisional application No. 61/987,660, filed on May 2, 2014.

(51) Int. Cl. C12Q 1/68; A61K 45/06; C07K 16/28; A61K 39/395; G01N 33/574; C07K 16/30

(52) U.S. Cl. CPC C12Q 1/6886; G01N 33/57419; C07K 16/3046; C07K 16/2863; A61K 39/39558; A61K 45/06; C12Q 2600/118; G01N 2333/485; G01N 2333/71; G01N 2333/912; G01N 2333/916; C12Q 2600/106; C12Q 2600/158; C07K 2317/24; C07K 2317/76

(57) ABSTRACT

The present disclosure provides biomarkers, and methods of using such biomarkers, that are predictive for efficacy of and resistance to an EGFR targeting agent, such as cetuximab, or prognostic with respect to cancer survival.

### CROSS-REFERENCE TO RELATED APPLICATIONS

This patent application claims the benefit of priority of U.S. Provisional Patent Application No. 61/987,660, filed May 2, 2014, which is incorporated herein by reference in its entirety.

### INTRODUCTION

Epidermal Growth Factor Receptor (EGFR) targeted therapies have shown clinical benefit in the treatment of numerous cancers, including metastatic colorectal cancer. The chimeric monoclonal IgG1 anti-EGFR antibody, cetuximab, has shown efficacy as monotherapy and in combination with irinotecan in late-line treatment (Cunningham et al., 2004; Jonker et al., 2007); however efficacy in first-line treatment of mCRC in combination with chemotherapy has only shown modest results (Van Cutsem et al., 2009; Van Cutsem, 2007). Cetuximab acts by binding the extracellular domain of EGFR thereby inhibiting ligand binding and dimerization, leading to internalization and degradation of the receptor. EGFR signals transduce through the Ras/Raf/Mek and PI3K/AKT/mTOR pathways to affect cancer cell proliferation, apoptosis, invasion, and angiogenesis.

Much effort has been devoted to identifying biomarkers that can predict patients most likely to benefit from EGFR-targeted therapies. Despite the fact that EGFR is overexpressed in 50-80% of colorectal tumors, protein expression as assessed by immunohistochemistry does not predict for clinical outcome in patients treated with anti-EGFR therapies (Chung et al., 2005; Hecht et al., 2010), although gene copy number has shown some association with clinical benefit (Laurent-Puig et al., 2009a; Moroni et al., 2005; Sartore-Bianchi et al., 2007; Scartozzi et al., 2009). Activating mutations within codons 12 or 13 of the KRAS gene have been shown to be predictive of resistance to anti-EGFR therapies (Amado et al., 2007; De Roock et al., 2010; De Roock et al., 2008; Di Fiore et al., 2007; Lievre et al., 2006). The KRAS gene is mutated in ˜40% of colorectal cancers (Bos et al., 1987); despite this, still only 10-40% of KRAS wildtype patients respond to cetuximab (Allegra et al., 2009). Mutations of other genes within the EGFR signaling pathway (BRAF, NRAS, PI3K, loss of PEN expression) have been shown to be rare or act as negative prognostic factors in mCRC, but it is unclear if these may act as predictive markers to cetuximab response (De Roock et al., 2010; Di Nicolantonio et al., 2008; Erben et al., 2011; Laurent-Puig et al., 2009a; Laurent-Puig et al., 2009b; Li et al., 2010; Loupakis et al., 2009; Modest et al., 2012; Prenen et al., 2009; Roth et al., 2010; Sun et al., 2012). Additionally, high expression levels of two EGFR ligands, AREG and EREG, have been associated with longer progression free survival (PFS) and response rates in KRAS wildtype mCRC patients treated with cetuximab (Baker et al., 2011; Jacobs et al., 2009; Khambata-Ford et al., 2007). However, most of these biomarker studies for cetuximab treatment in mCRC patients have been performed in non-randomized clinical studies. Non-randomized studies cannot distinguish between prognostic and predictive markers. Prognostic effects can confound detection of the predictive markers, and vise versa. For these reasons, studies using randomized controlled studies are needed to investigate and validate potential new predictive or prognostic markers useful in treating cancer with, for example, EGFR-targeted therapies.

### SUMMARY

Provided herein are methods of predicting responsiveness of a cancer in a subject to a cancer therapy including a EGFR targeting agent, methods of developing a prognosis for a subject diagnosed with colorectal cancer, and methods of developing treatment regimens for subjects with cancer.

In one aspect, methods of predicting responsiveness of a cancer in a subject to a cancer therapy including a EGFR targeting agent are provided. Such methods comprise obtaining a biological sample from the subject; measuring an expression level of at least one biomarker selected from CD73, HER3, EGF, EGFR, HB-EGF, BTC, HER2, HER4, and DUSP4 in the sample from the subject; generating a comparison of the expression level of the biomarker in the sample to a reference level of the biomarker; and using said comparison to predict the responsiveness of the cancer to treatment with the cancer therapy including a EGFR targeting agent.

In another aspect, methods of developing a prognosis for a subject diagnosed with cancer are provided. Such methods comprise obtaining a biological sample from the subject; measuring an expression level of at least one biomarker selected from CD73, HER2, EREG, EGF, EGFR, HB-EFG, and HER3 in the sample from the subject; generating a comparison of the expression level of the biomarker in the sample to a reference level of the biomarker; and using said comparison to determine a survival prognosis for the subject.

In a further aspect, methods of treating cancer in a subject are provided. Such methods, comprise having determined an expression level of at least one biomarker selected from CD73, HER3, EGF, EGFR, HB-EGF, BTC, HER2, HER4, and DUSP4 in a biological sample from the subject; selecting a treatment regimen for the subject based on the expression of at least one of the biomarkers, and administering a therapeutically effective amount of an EGFR targeting agent in the subject if the cancer is predicted to be responsive to the EGFR targeting agent.

### BRIEF DESCRIPTION OF THE DRAWINGS

FIG. 1 is a consort diagram showing patient enrollment numbers and groups.

FIG. 2 is a set of forest plots showing associations of gene expression levels with OS in KRAS-WT (FIG. 2A) and KRAS-Mut (FIG. 2B) pts. All assayed genes are shown. The length of the line indicates the 95% confidence interval and the diameter of the median dot is inversely proportional to the standard deviation.

FIG. 3 is a set of forest plots showing associations of gene expression levels with PFS in KRAS-WT (FIG. 3A) and KRAS-Mut (FIG. 3B) pts. All assayed genes are shown. The length of the line indicates the 95% confidence interval and the diameter of the median dot is inversely proportional to the standard deviation.

FIG. 4 is a set of forest plots showing the associations of gene expression and treatment group with OS in KRAS-WT (FIG. 4A) and KRAS-Mut (FIG. 4B) patients. Only genes with P_{interaction}≦0.2 are shown. The length of the line indicates the 95% CI, and the diameter of the median dot is inversely proportional to the standard deviation.

FIG. 5 is a set of forest plots showing the associations of gene expression and treatment group with PFS in KRAS-WT (FIG. SA) and KRAS-Mut (FIG. 5B) patients. Only genes with P_{interaction}≦0.2 are shown. The length of the line indicates the 95% CI, and the diameter of the median dot is inversely proportional to the standard deviation.

FIG. 6 is a set of forest plots showing associations of gene expression levels and treatment group with OS in KRAS-WT (FIG. 6A) and KRAS-Mut (FIG. 6B) pts. All assayed genes are shown. The length of the line indicates the 95% confidence interval and the diameter of the median dot is inversely proportional to the standard deviation.

FIG. 7 is a set of forest plots showing associations of gene expression levels and treatment group with PFS in KRAS-WT (FIG. 7A) and KRAS-Mut (FIG. 7B) pts. All assayed genes are shown. The length of the line indicates the 95% confidence interval and the diameter of the median dot is inversely proportional to the standard deviation.

FIG. 8 is a set of Kaplan-Meier plots of tumor gene expression levels significantly associated with outcome. OS by HER3 expression in KRAS-WT patients (FIG. 8A), PFS by CD73 expression in KRAS-WT patients (FIG. 8B), PFS by CD73 expression in KRAS-Mut patients (FIG. 8C; all groups dichotomized at the median). P_{interaction} values are shown.

FIG. 9 is a flowchart showing patients selected for serum marker testing.

FIG. 10 is a set of prognostic forest plots show the association of each marker with OS (FIG. 10A, FIG. 10C, and FIG. 10E) or PFS (FIG. 10B, FIG. 10D, and FIG. 10F) for all patients (FIG. 10A and FIG. 10B) KRAS-WT patients (FIG. 10C and FIG. 10D) and KRAS-Mut patients (FIG. 10E and FIG. 10F).

FIG. 11 is a forest plot showing the association and the related statistics including the number of samples analyzed, the Hazard ratio calculated and the 95% confidence interval for each of the indicated biomarkers either above or below the median in all patients.

FIG. 12 is a forest plot showing the association and the related statistics including the number of samples analyzed, the Hazard ratio calculated and the 95% confidence interval for each of the indicated biomarkers either above or below the median in KRAS wild-type patients.

FIG. 13 is a forest plot showing the association and the related statistics including the number of samples analyzed, the Hazard ration calculated and the 95% confidence interval for each of the indicated biomarkers either above or below the median in KRAS mutant patients.

FIG. 14 is a set of Kaplan-Meier curves showing the effects of EGF level and treatment arm. FIG. 14A shows OS KRAS-WT patients (interaction p=0.045); FIG. 14B shows OS in KRAS-Mut patients (interaction p=0.026); and FIG. 14C shows PFS in KRAS-Mut patients (interaction p=0.001). High and low marker levels are dichotomized at an empirically-determined optimal cut-point.

FIG. 15 is a set of Kaplan-Meier curves showing the effects of sHer3 level and treatment arm. FIG. 15A shows OS in all patients (interaction p=0.046); and FIG. 15B shows PFS in all patients (interaction p=0.032). High and low marker levels are dichotomized at an empirically-determined optimal cut-point.

FIG. 16 is a set of Kaplan-Meier curves showing the effects of CD73 level and treatment arm. FIG. 16A shows OS KRAS-WT patients (interaction p=0.049); FIG. 16B shows PFS in KRAS-WT patients (interaction p=0.018); and FIG. 16C shows PFS in KRAS-Mut patients (interaction p=0.017). High and low marker levels are dichotomized at an empirically-determined optimal cut-point.

### DETAILED DESCRIPTION

The present disclosure is based on the finding that the RNA and protein expression of several genes/biomarkers, including EGF-signaling related biomarkers, in cancers and particularly in colorectal tumors are predictive for EGFR targeting agent efficacy and resistance and/or are prognostic for overall survival (OS) and/or progression free survival (PFS). There is a substantial need for the identification of such biomarkers to both improve outcomes for patients who receive EGFR targeting agents and reduce the negative outcomes associated with the futile treatment of patients unlikely to derive benefit from administration of EGFR targeting agents.

Methods of predicting responsiveness of a cancer in a subject to a cancer therapy including a EGFR targeting agent, methods of developing a prognosis for a subject diagnosed with cancer, in particular colorectal cancer, and methods of developing treatment regimens for subjects with cancer are provided herein. The methods all rely on detecting or determining the expression level of at least one biomarker or combinations of biomarkers in a sample from a subject diagnosed with cancer.

Thus, the present methods permit the personalization of therapy amongst cancer patients, wherein a subject's biomarker profile is predictive of, or indicative of, treatment efficacy with an EGFR targeting agent and/or prognostic of a survival measure. The methods disclosed herein can be used in combination with assessment of conventional clinical factors, such as tumor size, tumor grade, lymph node status, family history, and analysis of expression level of additional biomarkers. In this manner, the methods of the present disclosure permit a more accurate prediction of cancer therapy effectiveness and/or evaluation of prognosis.

In one aspect, the method includes measuring or having determined an expression level of at least one of the biomarkers selected from: CD73, HER3, EGF, EGFR, HB-EGF, BTC, HER2, HER4, and DUSP4 in a sample from the subject. CD73 is an extracellular 5′ ectonucleotidase. HER3 is a member of the EGFR/HER family of receptor tyrosine kinases. EGF (epidermal growth factor) is a growth factor that binds to EGFR (epidermal growth factor receptor). EGFR is also known as ErbB-1 or HER1. HB-EGF (heparin-binding EGF-like growth factor) is a member of the EGF family of proteins. BTC (betacellulin) is a member of the EGF family. HER2 (human epidermal growth factor receptor 2) is a member of the EGFR family and is also known as ErbB-2. HER4 (human epidermal growth factor receptor 4) is a member of the EGFR family and is also known as ErbB-4. DUSP4 (dual specificity phosphatase 4) is an enzyme.

In another aspect, the method includes developing a prognosis for a subject diagnosed with cancer by measuring an expression level of at least one biomarker selected from CD73, HER2, EREG, EGF, EGFR, HB-EGF, and HER3. EREG (epiregulin) is a member of the EGF family of proteins.

In one embodiment, the method includes predicting responsiveness of a cancer in a subject to a cancer therapy including a EGFR targeting agent by obtaining a biological sample from the subject and measuring an expression level of at least one biomarker selected from CD73, HER3, EGF, EGFR, HB-EGF, BTC, HER2, HER4, and DUSP4 in the sample from the subject. The expression level of the biomarker in the sample is then compared to a reference level of the biomarker and this comparison is used to predict the responsiveness of the cancer to treatment with the cancer therapy including a EGFR targeting agent.

In some embodiments, the methods further include administering an EGFR targeting agent to a subject if the cancer is predicted to be responsive to the EGFR targeting agent. The methods of the present disclosure generally comprise administering to a subject (e.g., a human) a compound or a composition or therapy disclosed herein. Such administering can be local administration or systemic administration. The EGFR targeting agents may be administered by any means known to those skilled in the art, including, but not limited to, oral, topical, intranasal, intraperitoneal, parenteral, intravenous, intramuscular, subcutaneous, intrathecal, transcutaneous, nasopharyngeal, or transmucosal absorption. Thus the compounds may be formulated as an ingestable, injectable, topical or suppository formulation or a part of an implant. The compounds may also be delivered within a liposomal or time-release vehicle. Administration of the EGFR targeting agent, alone or in combination with another cancer therapeutic agent will be in an effective amount, which may be defined as an amount effective to treat the cancer.

Treating cancer includes, but is not limited to, reducing the number of cancer cells or the size of a tumor in the subject, reducing progression of a cancer to a more aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer, including improvement in the condition of the subject (e.g., in one or more symptoms), delay in the progression of the disease, delay in the onset of symptoms or slowing the progression of symptoms, an increase in the length of progression free survival or an increase in the overall survival of the subject after treatment.

As used herein the term “predicting responsiveness” refers to providing a probability based analysis of how a particular subject will respond to a cancer therapy. The prediction of responsiveness is not a guarantee or absolute, only a statistically probable indication of the responsiveness of the subject. The prediction of responsiveness to a cancer therapy including a EGFR targeting agent may indicate that the subject is likely to be responsive to a cancer therapy including a EGFR targeting agent or alternatively may indicate that the subject is not likely to be responsive to a cancer therapy including a EGFR targeting agent. Alternatively, the prediction may indicate that inclusion of a EGFR targeting agent in a treatment regimen may be counter-productive and lead to a worse result for the subject than if no therapy was used or a placebo was used. Responsiveness includes, without limitation, any measure of a likelihood of clinical benefit. For example, clinical benefits include an increase in overall survival, an increase in progression free survival, an increase in time to progression, increased tumor response, decreased symptoms, or other quality of life benefits.

As used herein, the term “subject” and “patient” are used interchangeably and refer to both human and non-human animals. The term “non-human animals” of the disclosure includes all vertebrates, e.g., mammals and non-mammals, such as nonhuman primates, sheep, dog, cat, horse, cow, chickens, amphibians, reptiles, and the like. Preferably, the subject is a human patient. More preferably, the subject is a human patient diagnosed with cancer or undergoing, or about to undergo, a cancer treatment regimen. A human subject may also have a genotype that is “wildtype” or “mutant” at the KRAS gene, KRAS-WT or KRAS-Mut respectively. For example, subjects with a KRAS-Mut genotype may have seven common mutations of the KRAS gene at codons 12 and 13 (G12A, G12D, G12R, G12C, G12S, G12V, and G13D).

As used herein, the term “subject diagnosed with cancer” refers to a subject that presents one or more symptoms indicative of a cancer (e.g., a noticeable lump or mass) or has been diagnosed as having cancer.

The cancer may be selected from any cancer in which a EGFR targeting agent is being considered for therapeutic purposes. The cancer may be a solid tumor. Cancers for which predictions may be made include, without limitation, colorectal, pancreatic, breast, liver, esophageal, gastric, kidney, small bowel, cholangiocarcinoma, lung, head and neck, thyroid, melanoma, breast, renal, bladder, ovarian, cervical, uterine, prostate, lymphomas, leukemias, neuroendocrine, glioblastoma or any other form of brain cancer. Preferably, the cancer is colorectal cancer. The colorectal cancer may be a metastatic colorectal cancer.

A EGFR targeting agent includes any therapeutic agent targeting any member of the EGFR family of proteins. In particular, antibodies specific for EGFR or other bioreagents capable of affecting EGFR mediated signaling, such as EGFR binding or competitive inhibitors, small molecules, aptamers, iRNAs, siRNAs, microRNAs, and other non-antibody-based therapeutic reagents.

EGFR targeting agents include, but are not limited to cetuximab (Erbitux™), gefitinib (Iressa™), erlotinib (Tarceva™), afatinib (Gilotrif™), brigatinib, and icotinib. Cetuximab is a monoclonal antibody against EGFR that is FDA approved for the treatment of head and neck cancer and colorectal cancer in patients whose tumors have a KRAS-wildtype gene. Gefitinib, Erlotinib, Afatinib, Brigatinib, and Icotinib are all small molecule inhibitors of EGFR each used to treat certain types of cancer. Multiple other EGFR targeting agents are in various stages of clinical development. Preferably, the EGFR targeting agent is cetuximab.

EGFR targeting agents may be used in combination with other cancer therapeutics in a cancer therapy. Combination therapy does not require that multiple cancer therapeutics be administered simultaneously, but only that the subjects are treated with more than one therapeutic agent during a time span, such as one month, two months or more. In some embodiments, the cancer therapy includes a chemotherapy agent in addition to the EGFR targeting agent. In the Examples, patients where treated with a combination therapy that included FOLFOX or FOLFIRI with or without cetuximab. FOLFOX is a chemotherapy regimen made up of the drugs folinic acid (leucovorin), fluorouracil (5-FU), and oxaliplatin. FOLFIRI is a chemotherapy regimen made up of the drugs folinic acid (leucovorin), fluorouracil (5-FU), and irintecan. The EGFR targeting agent and the cancer therapeutics may be administered in any order, at the same time or as part of a unitary composition. The two inhibitors may be administered such that one inhibitor is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

In particular embodiments, the methods for predicting responsiveness of a cancer to a cancer therapy or developing a prognosis for a subject diagnosed with a cancer includes obtaining or having analyzed a biological sample from a subject. The sample may or may not include cells. In particular, the methods described herein may be performed without requiring a tissue sample or biopsy and need not contain any cancer cells. In the Examples, a blood sample (such as a plasma sample) or biopsy sample (such as a tissue/cell sample) were used. “Sample” is intended to include any sampling of cells, tissues, or bodily fluids in which expression of a biomarker can be detected. Examples of such samples include, without limitation, biopsies, smears, blood, lymph, urine, saliva, or any other bodily secretion or derivative thereof. Blood can include whole blood, plasma (citrate, EDTA, heparin), serum, or any derivative of blood. Samples may be obtained from a subject by a variety of techniques available to those skilled in the art. Methods for collecting various samples are well known in the art. In some embodiments, the biological sample is a blood or plasma sample. In some embodiments, the biological sample is a tumor sample or cancer cells.

A “biomarker” is a nucleic acid, protein, or other chemical whose level of expression in a sample is indicative of a condition. In the Examples, the biomarkers are expression levels of mRNA transcripts and/or proteins encoded by genes. In some embodiments, the expression level of the biomarker is the protein expression level. In some embodiments, the expression level of the biomarker is the mRNA expression level. These expression levels have been found to correlate with responsiveness of the cancer to a cancer therapy including a EGFR targeting agent and/or prognosis for a subject diagnosed with cancer.

Biomarker expression in some instances may be normalized against the expression levels of all proteins or RNA transcripts in the sample, or against a reference set of proteins or RNA transcripts in the sample. The level of expression of the biomarkers is indicative of the prognosis for the subject or predictive of the effectiveness of a particular treatment.

Fragments and variants of biomarker mRNA transcripts and proteins are also encompassed by the present invention. By “fragment” is intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs.

Any methods available in the art for detecting expression of biomarkers are encompassed herein. The expression of a biomarker of the invention can be detected on a nucleic acid level (e.g., as an mRNA transcript) or a protein level. “Measuring the expression level” means determining the quantity or presence of a protein or its RNA transcript for at least one of the biomarkers disclosed herein. Thus, “measuring the expression level” encompasses instances where a biomarker is determined not to be expressed, not to be detectably expressed, expressed at a low level, expressed at a normal level, or overexpressed. The expression level may be measured relative to a control.

“Having determined the expression level” means that a person may request that the expression level of the biomarkers be determined by a laboratory using any of the methods known in the art or disclosed herein. The laboratory may be part of the organization that employs the person or the laboratory may reside in an entity not associated with the person such as commercial laboratory. For example, a physician may have the expression level of one of the biomarkers disclosed herein determined by requesting such measurements be performed by a laboratory that is or is not associated with the physician.

Methods suitable for measuring, detecting, or determining the expression levels of biomarkers are known to those of skill in the art and include, but are not limited to, ELISA, immunofluorescence, FACS analysis, Western blot, magnetic immunoassays, and both antibody-based microarrays and non-antibody-based microarrays. In the past, the gold standard for detection of growth factors and cytokines in blood was the use of ELISAs; however, multiplex technology offers an attractive alternative approach for cytokine and growth factor analysis. The advantages of multiplex technology compared to traditional ELISA assays are conservation of patient sample, increased sensitivity, and significant savings in cost, time and labor.

Several multiplex platforms currently exist. The Luminex bead-based systems are the most established, being used to detect circulating cytokines and growth factors in both mice and humans. This method is based on the use of microparticles that have been pre-coated with specific antibodies. These particles are then mixed with sample and the captured analytes are detected using specific secondary antibodies. This allows for up to 100 different analytes to be measured simultaneously in a single microplate well. The advantages of this flow cytometry-based method compared to traditional ELISA assays are in the conservation of patient samples as well as significant savings in terms of cost and labor. An alternative, plate-based system is produced by Meso Scale Discovery (MSD). This system utilizes its proprietary Multi-Array® and Multi-Spot® microplates with electrodes directly integrated into the plates. This enables the MSD system to have ultra-sensitive detection limits, high specificity, and low background signal. Another plate-based multiplex system is the SearchLight Plus CCD Imaging System produced by Aushon Biosystems. This novel multiplexing technology allows for the measurement of up to 16 different analytes simultaneously in a single microplate well. The assay design is similar to a sandwich ELISA where the capture antibodies are pre-spotted into individual wells of a 96-well plate. Samples or standards are added which bind to the specific capture antibodies and are detected using Aushon's patented SuperSignal ELISA Femto Chemiluminescent Substrate.

Methods for detecting expression of the biomarkers described herein are not limited to protein expression. Gene expression profiling including methods based on hybridization analysis of polynucleotides, methods based on sequencing of polynucleotides, immunohistochemistry methods, and proteomics-based methods may also be used. The most commonly used methods known in the art for the quantification of mRNA expression in a sample include northern blotting and in situ hybridization (Parker and Barnes, Methods Mol. Biol. 106:247-83, 1999), RNAse protection assays (Hod, Biotechniques 13:852-54, 1992), PCR-based methods, such as reverse transcription PCR(RT-PCR) (Weis et al., TIG 8:263-64, 1992), including real time quantitative PCR and array-based methods (Schena et al., Science 270:467-70, 1995). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE) and gene expression analysis by massively parallel signature sequencing.

In the methods described herein the expression level of at least one biomarker described herein in a sample from the subject is determined using any one of the detection methods described herein. Then the level in the sample from the subject is compared to a reference level of the biomarker or a control. The “reference level” may be determined empirically such as it was in the Examples, by comparison to the levels found in a set of samples from cancer patients treated with cancer therapies including or excluding a EGFR targeting agent with known clinical outcomes for the patients. Alternatively, the reference level may be a level of the biomarker found in samples, such as plasma samples, which becomes a standard and can be used as a predictor for new samples. For example, the median cut-off levels reported in the Examples or such median values altered by 5%, 10%, 20%, 30%, 40%, 50%, 60%, 70%, 80%, or 90% may now serve as reference levels for comparison.

In one embodiment, the protein expression level of CD73 is determined in a blood or plasma sample from a subject to generate a prediction. In this embodiment, the prediction indicates responsiveness to a EGFR targeting agent when the protein expression level of CD73 is more than 4.3, 10, 20, 30, 40, 50, or 60 ng/mL.

In another embodiment, the protein expression level of HER3 is determined in a blood or plasma sample from a subject to generate a prediction. In this embodiment, the prediction indicates responsiveness to a EGFR targeting agent when the protein expression level of HER3 is more than 11, 15, 20, 25, 30, 35, or 40 ng/mL.

In another embodiment, the protein expression level of EGF is determined in a blood or plasma sample from a subject that is KRAS-WT to generate a prediction. In this embodiment, the prediction indicates lack of responsiveness to a EGFR targeting agent when the protein expression level of EGF is more than 19.8, 40, 60, 80, 100, 120, 140, 160, 180, 200, 220, 240, 260, 280, 300, 320, or 340 pg/mL.

In another embodiment, the protein expression level of EGF is determined in a blood or plasma sample from a subject that is KRAS-Mut to generate a prediction. In this embodiment, the prediction indicates responsiveness to a EGFR targeting agent when the protein expression level of EGF is more than 19.8, 40, 60, 80, 100, 120, 140, 160, 180, 200, 220, 240, 260, 280, 300, 320, or 340 pg/mL.

In another embodiment, the protein expression level of EGFR is determined in a blood or plasma sample from a subject that is KRAS-Mut to generate a prediction. In this embodiment, the prediction indicates responsiveness to a EGFR targeting agent when the protein expression level of EGFR is more than 25.6, 26, 28, 30, 32, 35, 37, or 40 ng/mL.

In another embodiment, the mRNA expression level of CD73 is determined in a tumor sample or cancer cells from a subject to generate a prediction. In this embodiment, the prediction indicates responsiveness to a EGFR targeting agent when the mRNA expression level of CD73 is higher than a reference level.

In another embodiment, the mRNA expression level of HER3 is determined in a tumor sample or cancer cells from a subject that is KRAS-WT to generate a prediction. In this embodiment, the prediction indicates lack of responsiveness to a EGFR targeting agent when the mRNA expression level of HER3 is higher than a reference level.

In another embodiment, the mRNA expression level of BTC is determined in a tumor sample or cancer cells from a subject that is KRAS-WT to generate a prediction. In this embodiment, the prediction indicates lack of responsiveness to a EGFR targeting agent when the mRNA expression level of BTC is higher than a reference level.

In another embodiment, the mRNA expression level of HER4 is determined in a tumor sample or cancer cells from a subject to generate a prediction. In this embodiment, the prediction indicates responsiveness to a EGFR targeting agent when the mRNA expression level of HER4 is higher than a reference level.

In another embodiment, the mRNA expression level of DUSP4 is determined in a tumor sample or cancer cells from a subject that is KRAS-WT to generate a prediction. In this embodiment, the prediction indicates responsiveness to a EGFR targeting agent when the mRNA expression level of DUSP4 is higher than a reference level.

In another embodiment, the mRNA expression level of HER2 is determined in a tumor sample or cancer cells from a subject that is KRAS-Mut to generate a prediction. In this embodiment, the prediction indicates lack of responsiveness to a EGFR targeting agent when the mRNA expression level of HER2 is higher than a reference level.

In another embodiment, the mRNA expression level of HB-EGF is determined in a tumor sample or cancer cells from a subject that is KRAS-Mut to generate a prediction. In this embodiment, the prediction indicates lack of responsiveness to a EGFR targeting agent when the mRNA expression level of HB-EGF is higher than a reference level.

In one embodiment, the method includes developing a prognosis for a subject diagnosed with cancer comprising: obtaining a biological sample from the subject; measuring an expression level of at least one biomarker selected from CD73, HER2, EREG, EGF, EGFR, HB-EFG, and HER3 in the sample from the subject; generating a comparison of the expression level of the biomarker in the sample to a reference level of the biomarker; using said comparison to determine a survival prognosis for the subject.

As used herein, “survival prognosis” indicates some measure of subject survival including, without limitation, overall survival or longer progression free survival.

In one embodiment, the protein expression level of CD73 is determined in a blood or plasma sample from a subject to generate a prognosis. In this embodiment, an expression level of CD73 less than 4.3, 3, 2, or 1 ng/mL is indicative of a better prognosis.

In one embodiment, the protein expression level of HER2 is determined in a blood or plasma sample from a subject that is KRAS-Mut to generate a prognosis. In this embodiment, an expression level of HER2 greater than 3.2, 3.5, 4, 5, 6, 7, 10, 12, 15, 17, or 20 ng/mL is indicative of a better prognosis.

In one embodiment, the protein expression level of EGF is determined in a blood or plasma sample from a subject to generate a prognosis. In this embodiment, an expression level of EGF less than 19.8, 17, 15, 13, 11, 9, 7, 5, 3, or 1 pg/mL is indicative of a better prognosis.

In one embodiment, the protein expression level of EGFR is determined in a blood or plasma sample from a subject that is KRAS-Mut to generate a prognosis. In this embodiment, an expression level of EGFR greater than 25.6, 26, 28, 30, 32, 35, 37, or 40 ng/mL is indicative of a better prognosis.

In one embodiment, the protein expression level of HER3 is determined in a blood or plasma sample from a subject to generate a prognosis. In this embodiment, an expression level of HER3 less than 11, 9, 7, 5, 3, or 1 ng/mL is indicative of a better prognosis.

In one embodiment, the protein expression level of HB-EGF is determined in a blood or plasma sample from a subject to generate a prognosis. In this embodiment, an expression level of HB-EGF less than 14.8, 12, 10, 8, 6, 4, or 2 pg/mL is indicative of a better prognosis.

In one embodiment, the mRNA expression level of HER2 is determined in a tumor sample or cancer cells from a subject to generate a prognosis. In this embodiment, an expression level of HER2 greater than a reference level is indicative of a better prognosis.

In one embodiment, the mRNA expression level of EGF is determined in a tumor sample or cancer cells from a subject to generate a prognosis. In this embodiment, an expression level of EGF greater than a reference level is indicative of a better prognosis.

In one embodiment, the mRNA expression level of EREG is determined in a tumor sample or cancer cells from a subject that is KRAS-WT to generate a prognosis. In this embodiment, an expression level of EREG greater than a reference level is indicative of a better prognosis.

As used herein, “better prognosis” means the subject has a longer survival by some defined measure. In some embodiments, a better prognosis indicates longer overall survival or longer progression free survival as compared to controls. The term better is in comparison to subjects found to express the biomarkers at levels not correlated with an increase in survival.

In one embodiment, the method includes having determined an expression level of at least one biomarker selected from CD73, HER3, EGF, EGFR, HB-EGF, BTC, HER2, HER4, and DUSP4 in a biological sample from the subject; selecting a treatment regimen for the subject based on the expression of at least one of the biomarkers, and administering a therapeutically effective amount of an EGFR targeting agent in the subject if the cancer is predicted to be responsive to the EGFR targeting agent.

As used herein, “treatment regimen” or “treatment” refers to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. For example, such therapies may include surgery, medications (hormonal therapy and/or chemotherapy), radiation, immunotherapy and the like. Such treatments are well known and particular to the patient and can be readily determined by one skilled in the art. In some embodiments, the treatment regimen may comprise chemotherapy regimens such as FOLFOX or FOLFIRI with or without cetuximab. Such administration is specific to the subject and can be determined by one skilled in the art at the time of administration.

The term “effective amount” or “therapeutically effective amount” refers to an amount sufficient to effect beneficial or desirable biological and/or clinical results or to provide any level of treatment for the cancer.

In one embodiment, the biomarker comprises CD73 protein expression level measured in a blood or plasma sample from a subject. In this embodiment, the treatment regimen comprises a EGFR targeting agent when the protein expression level of CD73 is more than 4.3, 10, 20, 30, 40, 50, or 60 ng/mL.

In another embodiment, the biomarker comprises HER3 protein expression level measured in a blood or plasma sample from a subject. In this embodiment, the treatment regimen comprises a EGFR targeting agent when the protein expression level of HER3 is more than 11, 15, 20, 25, 30, 35, or 40 ng/mL.

In another embodiment, the biomarker comprises EGF protein expression level measured in a blood or plasma sample from a subject that is KRAS-WT. In this embodiment, the treatment regimen comprises a EGFR targeting agent when the protein expression level of EGF is less than 19.8, 40, 60, 80, 100, 120, 140, 160, 180, 200, 220, 240, 260, 280, 300, 320, or 340 pg/mL.

In another embodiment, the biomarker comprises EGF protein expression level measured in a blood or plasma sample from a subject that is KRAS-Mut. In this embodiment, the treatment regimen comprises a EGFR targeting agent when the protein expression level of EGF is more than 19.8, 40, 60, 80, 100, 120, 140, 160, 180, 200, 220, 240, 260, 280, 300, 320, or 340 pg/mL.

In another embodiment, the biomarker comprises EGFR protein expression level measured in a blood or plasma sample from a subject that is KRAS-Mut. In this embodiment, the treatment regimen comprises a EGFR targeting agent when the protein expression level of EGFR is more than 25.6, 26, 28, 30, 32, 35, 37, or 40 ng/mL.

In another embodiment, the biomarker comprises CD73 mRNA expression level measured in a tumor sample or cancer cells from a subject. In this embodiment, the treatment regimen comprises a EGFR targeting agent when the mRNA expression level of CD73 is higher than a reference level.

In another embodiment, the biomarker comprises HER3 mRNA expression level measured in a tumor sample or cancer cells from a subject that is KRAS-WT. In this embodiment, the treatment regimen comprises a EGFR targeting agent when the mRNA expression level of HER3 is lower than a reference level.

In another embodiment, the biomarker comprises BTC mRNA expression level measured in a tumor sample or cancer cells from a subject that is KRAS-WT. In this embodiment, the treatment regimen comprises a EGFR targeting agent when the mRNA expression level of BTC is lower than a reference level.

In another embodiment, the biomarker comprises HER4 mRNA expression level measured in a tumor sample or cancer cells from a subject. In this embodiment, the treatment regimen comprises a EGFR targeting agent when the mRNA expression level of HER4 is higher than a reference level.

In another embodiment, the biomarker comprises DUSP4 mRNA expression level measured in a tumor sample or cancer cells from a subject that is KRAS-WT. In this embodiment, the treatment regimen comprises a EGFR targeting agent when the mRNA expression level of DUSP4 is higher than a reference level.

In another embodiment, the biomarker comprises HER2 mRNA expression level measured in a tumor sample or cancer cells from a subject that is KRAS-Mut. In this embodiment, the treatment regimen comprises a EGFR targeting agent when the mRNA expression level of HER2 is lower than a reference level.

In another embodiment, the biomarker comprises HB-EGF mRNA expression level measured in a tumor sample or cancer cells from a subject that is KRAS-Mut. In this embodiment, the treatment regimen comprises a EGFR targeting agent when the mRNA expression level of HB-EGF is lower than a reference level.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

For the purposes of promoting an understanding of the principles of the present disclosure, reference will now be made to preferred embodiments and specific language will be used to describe the same. One skilled in the art will readily appreciate that the present invention is well adapted to carry out the objects and obtain the ends and advantages mentioned, as well as those inherent therein. The examples described herein are presently representative of preferred embodiments, are exemplary, and are not intended as limitations on the scope of the invention. Changes therein and other uses will occur to those skilled in the art which are encompassed within the spirit of the invention as defined by the scope of the claims.

The following examples are meant only to be illustrative and are not meant as limitations on the scope of the invention or of the appended claims. All references cited herein are hereby incorporated by reference in their entireties.

### EXAMPLES

### Example 1

### Gene Expression Markers of Efficacy and Resistance to Cetuximab Treatment in Metastatic Colorectal Cancer

### Patients and Methods

### Study Design and Patients

CALGB 80203 was a randomized phase II study of patients with colorectal cancer (CRC) treated using chemotherapy (either FOLFOX or FOLFIRI) with or without the addition of cetuximab. Cetuximab is a monoclonal antibody that binds epidermal growth factor receptor (EGFR) and competitively inhibits its interaction with epidermal growth factor (EGF). EGFR is overexpressed in 50-80% of colorectal tumors. This trial was designed to test whether the addition of cetuximab to chemotherapy regimens could improve treatment outcomes in CRC. Early findings of this study showed equivalent responses for FOLFIRI and FOLFOX therapies in first-line treatment of metastatic colorectal cancer (Venook, 2006). Preliminary results indicate an improved response rate of 52% in patients receiving cetuximab over 38% of patients that did not receive cetuximab (P=0.029)(Venook, 2006).

Patients with previously untreated, metastatic adenocarcinoma of the colon or rectum were randomized to FOLFIRI, FOLFIRI+cetuximab, FOLFOX, or FOLFOX+cetuximab treatment groups. This was a multi-center trial; 238 patients were randomized to treatment. Consent for biomarker analyses was optional. The protocol was approved by the institutional review boards at each participating institution. This retrospective analysis conforms to the reporting guidelines established by the REMARK criteria.

### Sample Collection

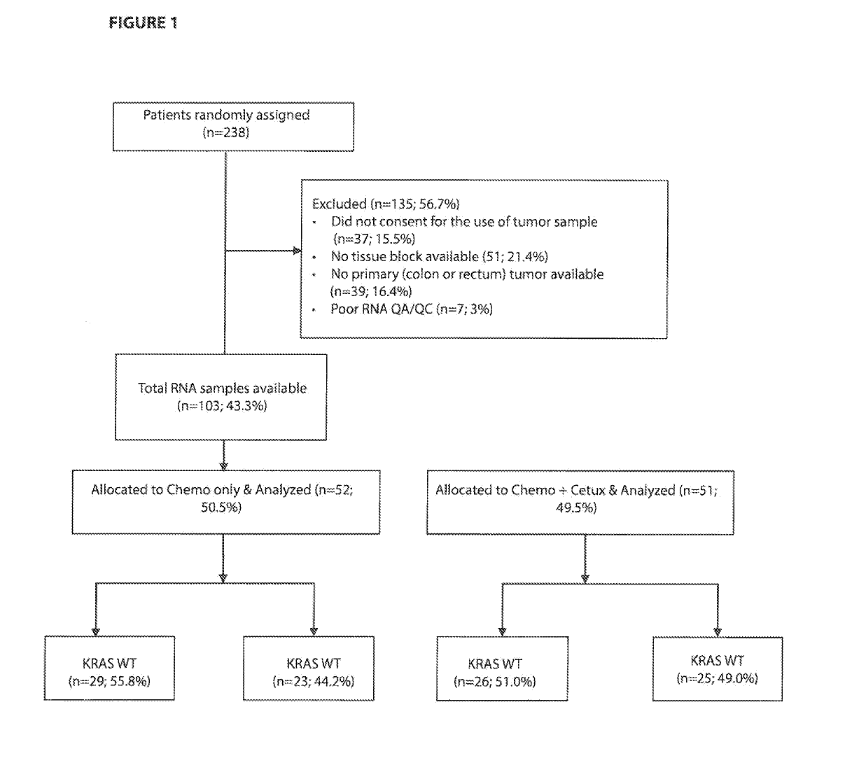

Formalin-fixed, paraffin embedded (“FFPE”) baseline tumor samples were collected during study enrollment. A total of 110 patients (48%) had at least one paraffin block of primary colon or rectum tumor available for analysis. Seven samples were further excluded from this analysis due to quality and quantity issues related to the RNA isolation (FIG. 1). All assays were performed in triplicate and all analysis was conducted while blinded to clinical outcome.

### KRAS Mutational Analysis

KRAS mutation status was determined by Real Time PCR using the TheraScreen: KRAS Mutation Test Kit from Qiagen-DxS Diagnostic Innovations, which is able to detect the seven common mutations of the KRAS gene at codons 12 and 13 (G12A, G12D, G12R, G12C, G12S, G12V, and G13D). Analysis was performed in the CALGB/Alliance molecular reference laboratory of Greg Tsongalis at Dartmouth Medical School.

### RNA Isolation and RT-qPCR Analysis

A hemolysin and eosin stained image of the tumor sample was reviewed by a pathologist to ensure the presence of >70% tumor tissue within the sample and quality of the tumor. If samples were <70% tumor, macro-dissection was performed manually. FFPE tumor biopsies were cut at the CALGB (Alliance) pathology coordinating office and shipped overnight to the Alliance molecular reference laboratory at Duke University. RNA was isolated from six 10-μm sections using the Ambion RecoverAll Total Nucleic Isolation kit according to manufacturer's protocol (Ambion-Life Technologies, Austin, Tex., USA). RNA (200 ng) from each sample was reverse transcribed using the High Capacity cDNA Reverse Transcription kit (Applied Biosystems-Life Technologies, Foster City, Calif., USA). Taqman quantitative PCR was performed for EGF-related gene expression (primer-probe sets described in Table 1), using the ABI 7900HT Real Time PCR System (Applied Biosystems-Life Technologies, Foster City, Calif., USA). The log transformed relative amounts of mRNA expression were normalized to β-actin mRNA and expressed as log 2^{-(CycleX-Cycleβ-actin)}=−(CycleX-Cycleβ-actin). Taqman gene expression assays were chosen for each gene to span exon-exon junctions and have small amplicons<100 base pairs to allow for specific and sensitive detection of partially degraded RNA. Life Technologies Taqman Gene Expression Assays have amplification efficiencies of ˜100% (+/−10%). The β-actin endogenous control was used in this analysis. We observed uniform expression of β-actin across the mCRC tumor samples in this study. The mean threshold cycle value was 23.6 cycles with a standard deviation of 1.9 cycles across the CALGB 80203 sample population. Duplicate samples with threshold cycle standard deviation greater than 0.5 cycles were re-run for improved qPCR reproducibility.

TABLE 1
List of assay primer sets used in this study.
All assay sets were purchased from Applied
Biosystems-Life Technologies, Foster City, CA, USA

| | | Samples With Detectable |
|---|---|---|
| Gene | Assay ID | Expression, N(%) |
| AREG | Hs00950669_m1 | 103 (100) |
| β-ACTIN | Hs00357333_g1 | 103 (100) |
| BTC | Hs01101201_m1 | 103 (100) |
| CD73 | Hs04234687_m1 | 95 (92) |
| DUSP4 | Hs01027785_m1 | 102 (99) |
| EGF | Hs00153181_m1 | 40 (39) |
| EGFR | Hs00193306_m1 | 103 (100) |
| EPGN | Hs02385428_m1 | 21 (20) |
| EREG | Hs00914313_m1 | 98 (95) |
| HBEGF | Hs00181813_m1 | 102 (99) |
| HER2 | Hs01001580_m1 | 103 (100) |
| HER3 | Hs00176538_m1 | 103 (99) |
| HER4 | Hs00955525_m1 | 32 (31) |
| PHILDA1 | Hs00378285_g1 | 103 (100) |
| TGFA | Hs00608187_m1 | 102 (99) |

The Taqman primer sets used were directed to RNA transcripts from the AREG, B-ACTIN, BTC, CD73, DUSP4, EGF, EGFR, EPGN, EREG, HBEGF HER2, HER3, HER4, PHIDA1, and WGFA genes.

### Statistical Analysis

Expression levels were normalized relative to β-actin, as described above, and analyzed as continuous measures. A Kendall tau analysis was performed to identify co-regulated genes. Univariate Cox regression was used to identify markers prognostic of clinical outcomes (Overall Survival (“OS”) and Progression Free Survival (“PFS”)), and the resulting p-values, hazard ratios, and 95% confidence intervals are reported. To identify predictive markers, expression level was correlated with clinical outcomes (OS and PFS) using multiplicative Cox proportional hazards models to test for interaction between genetic expression and treatment (chemo vs. chemo+cetuximab). Visualizations of the resulting effect sizes are provided in the form of forest plots. The forest plots illustrate the hazard ratios of the expression levels (and the corresponding 95% confidence interval) within each treatment group, and the p-values for the tests of interaction are provided. Kaplan-Meier plots of OS and PFS were generated as additional visualizations of selected predictive markers, with separate curves for each combination of treatment group and expression level (where expression level is dichotomized at the median as “high” or “low”). Analyses were conducted using all patients, as well as separately within KRAS-wildtype (“KRAS-WT”) and KRAS mutant (“KRAS-Mut”) subgroups, due to known differential responses to cetuximab across these populations. The reported p-values have not been adjusted for multiple testing. Data collection and statistical analyses were conducted by the Alliance Statistics and Data Center. All clinical data was locked on Mar. 5, 2012. Statistical analyses and figures were generated using the R software environment for statistical computing and graphic with the survival package.

### Results

### Patient Characteristics

Patients (238) with previously untreated mCRC were enrolled and randomly assigned to one of four treatment groups: FOLFOX, FOLFOX+cetuximab, FOLFIRI, or FOLFIRI+cetuximab. The FOLFOX and FOLFIRI treatment groups showed similar response rates, PFS and OS (Venook et al., 2006). Due to the small size of this study and similar outcomes across the FOLFOX and FOLFIRI treatment groups, these groups were combined into chemotherapy (chemo) only and chemo+cetuximab cohorts for this analysis. Patient characteristics of the biomarker population were similar to those of the overall population (Table 2). While most studies have indicated that KRAS exon 2 mutations comprise approximately 40% of the CRC patient population, the biomarker population in this study had a slightly higher proportion of KRAS Mut patients (Table 2). Within the biomarker population, the chemo+cetuximab cohort showed longer median PFS and OS times with higher response rates compared to the chemo only cohort, but these differences were not statistically significant.

FFPE tissue blocks from the primary tumor site (colon or rectum) were processed from 110 patients, however seven RNA samples were excluded due to RNA quality and quantity issues, leaving 103 patients (43%) to be included in this RNA biomarker analysis (FIG. 1). These patients were evenly distributed within the chemo only and chemo-cetuximab treatment groups (52 vs. 51 patients). The median follow-up time for all 103 patients included in the biomarker cohort was 69.2 months.

TABLE 2

| | | Overall whole | Overall biomarker | Chemo-only | Chemo + cetux |
|---|---|---|---|---|---|
| | | population | population | (biomarker population) | (biomarker population) |
| | | n (%) | n (%) | n (%) | n (%) |
| Patients | | 238 (100) | 103 (43) | 52 (50.5) | 51 (49.5) |
| Age, y | | | | | |
| | Median | 51.3 | 61.1 | [symbol] | 50.8 |
| | Range | 22-88.4 | 22-83.3 | 22-83.2 | 40.4-83.3 |
| | Gender male | 140 (58.9) | [symbol] | 27 (51.9) | 30 (58.8) |
| | Race white | 207 (87.0) | 91 (88.3) | [symbol] | 45 (90.2) |
| ECOS PS | | | | | |
| | 0 | 123 (82.5) | 51 (49.5) | 25 (48.1) | 26 (51) |
| | 1 | 113 (47.5) | [symbol] | 27 (51.9) | 25 (49) |

| | | | | |
|---|---|---|---|---|
| KRAS-WT | 84/165 (57) | 55 (53.4) | 29 (55.8) | 26 (51) |
| Median OS (99%Cl) | 23.0 (20.5-26.1) | 25.4 (22.5-32) | 22.8 (16.7-33) | 27.6 (23.4-38.0) |
| Median PFS (99%Cl) | 11.05 (9.73-13.04) | 9.67 (8.05-12.45) | 9.66 (8.34-12.6) | 10.25 (6.9-15.3) |
| Response rate (CR/PR) | 104 (43.7) | 42 (40.8) | 20 (38.5) | 22 (43.1) |

[symbol]

indicates data missing or illegible when filed

### Gene Expression in Primary Tumors

Expression of 14 genes related to the EGF-signaling pathway (AREG, BTC, CD73, DUSP4, EGF, EGFR, EPGN, EREG, HBEGF, HER2, HER3, HER4, PHLDA1, and TGFA) was analyzed using Taqman RT-qPCR from the primary tumors. Most genes were expressed at detectable levels in >90% patients (Table 3). Gene expression was most strongly correlated between EREG and AREG (t=0.553), with HER2 and HER3 also showing strong co-expression (t=0.475) (Table 3). EPGN was co-expressed with both HER4 (r=0.500) and EGF (r=0.571), but the low expression levels of these genes may affect interpretation of these results (Table 3).

TABLE 3
Table of co-regulation among the 14 genes using a Kendall tau analysis. The most highly associated genes are indicated in bold.

| | AREG | BTC | CD73 | DUSP4 | EGF | EGFR | EPGN | EREG | HBEGF | HER2 | HER3 | HER4 | PHLDA1 | TGFA |
|---|---|---|---|---|---|---|---|---|---|---|---|---|---|---|
| AREG | 1 | 0.322 | 0.137 | −0.026 | 0.151 | 0.250 | −0.038 | 0.553 | 0.216 | 0.146 | 0.193 | −0.024 | 0.242 | 0.048 |
| BTC | 0.322 | 1 | 0.159 | 0.128 | 0.164 | 0.265 | 0.057 | 0.209 | 0.171 | 0.365 | 0.369 | 0.113 | 0.245 | 0.045 |
| CD73 | 0.137 | 0.159 | 1 | 0.240 | 0.195 | 0.222 | −0.048 | 0.023 | −0.017 | 0.052 | 0.086 | 0.118 | 0.197 | 0.054 |
| DUSP4 | −0.026 | 0.128 | 0.240 | 1 | −0.108 | 0.066 | −0.152 | −0.181 | 0.245 | 0.163 | −0.021 | 0.308 | 0.394 | 0.148 |
| EGF | 0.151 | 0.164 | 0.195 | −0.108 | 1 | 0.205 | 0.571 | 0.061 | −0.195 | 0.036 | 0.123 | 0.390 | −0.187 | −0.223 |
| EGFR | 0.250 | 0.265 | 0.222 | 0.066 | 0.205 | 1 | 0.200 | 0.128 | 0.005 | 0.215 | 0.260 | 0.149 | 0.057 | 0.120 |
| EPGN | −0.038 | 0.057 | −0.048 | −0.152 | 0.571 | 0.200 | 1 | −0.011 | −0.238 | 0.076 | 0.190 | 0.500 | −0.114 | −0.410 |
| EREG | 0.553 | 0.209 | 0.023 | −0.181 | 0.061 | 0.128 | −0.011 | 1 | 0.152 | 0.097 | 0.154 | −0.041 | 0.127 | 0.025 |
| HBEGF | 0.216 | 0.171 | −0.017 | 0.245 | −0.195 | 0.005 | −0.238 | 0.152 | 1 | 0.117 | 0.088 | 0.213 | 0.302 | 0.223 |
| HER2 | 0.146 | 0.365 | 0.052 | 0.163 | 0.036 | 0.215 | 0.076 | 0.097 | 0.117 | 1 | 0.475 | 0.169 | 0.175 | 0.154 |
| HER3 | 0.193 | 0.369 | 0.086 | −0.021 | 0.123 | 0.260 | 0.190 | 0.154 | 0.088 | 0.475 | 1 | 0.093 | 0.133 | 0.104 |
| HER4 | −0.024 | 0.113 | 0.118 | 0.308 | 0.390 | 0.149 | 0.500 | −0.041 | 0.213 | 0.169 | 0.093 | 1 | 0.040 | −0.101 |
| PHLDA1 | 0.242 | 0.245 | 0.197 | 0.394 | −0.187 | 0.057 | −0.114 | 0.127 | 0.302 | 0.175 | 0.133 | 0.040 | 1 | 0.156 |
| TGFA | 0.048 | 0.045 | 0.054 | 0.148 | −0.223 | 0.120 | −0.410 | 0.025 | 0.223 | 0.154 | 0.104 | −0.101 | 0.156 | 1 |

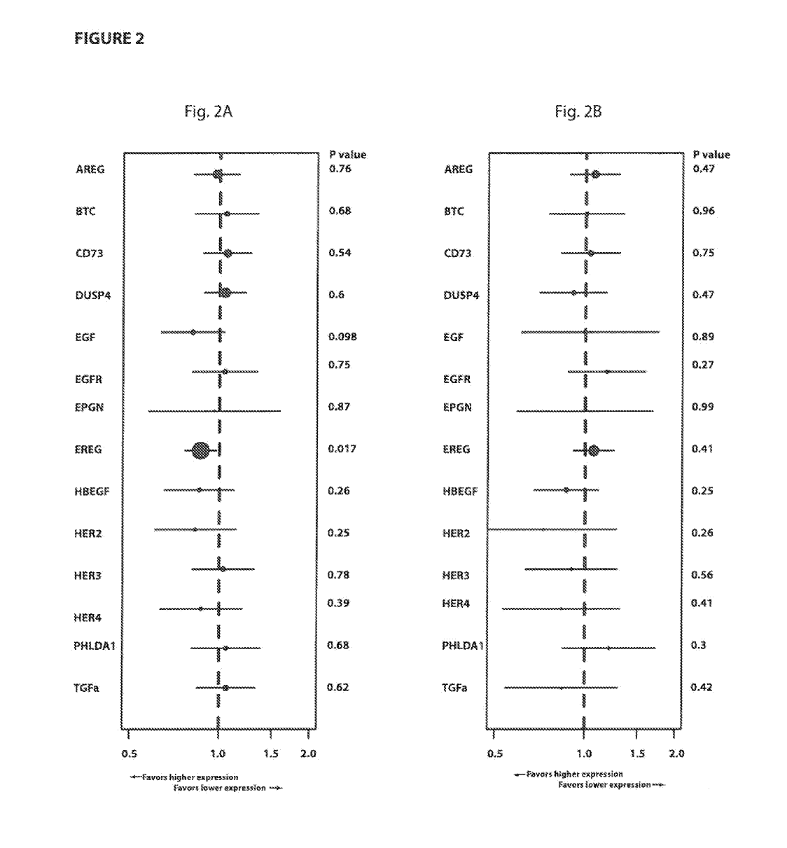

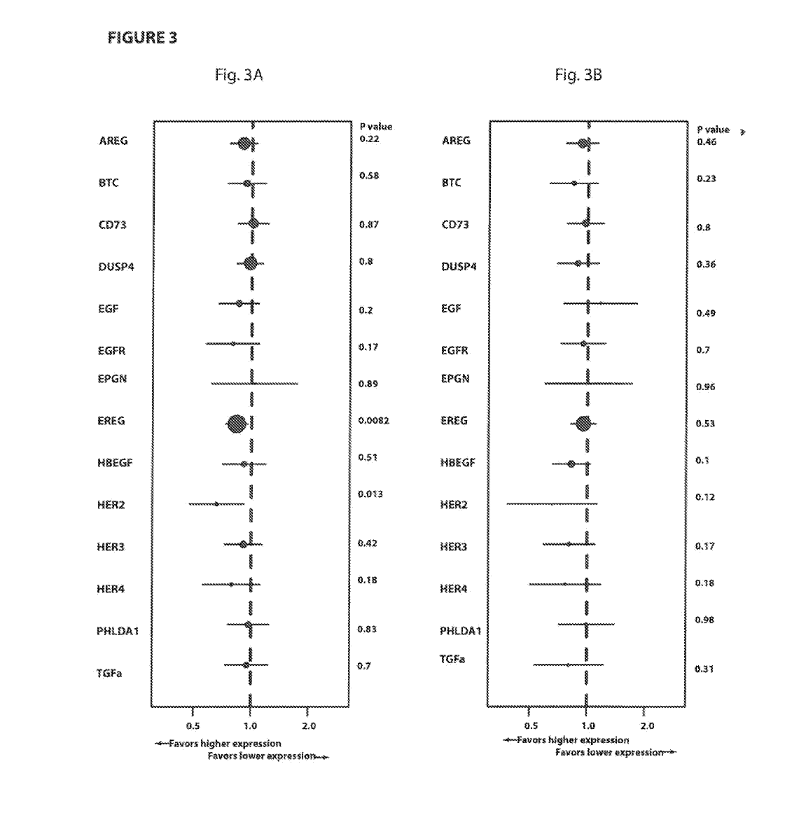

The baseline gene expression levels were tested for association with OS and PFS using Cox proportional hazards regression modeling. Prognostic univariate Cox regression analyses were conducted across all patients, and within KRAS-WT and KRAS-Mut subgroups. For OS across all patients, none of the assayed genes were identified as statistically significant prognostic markers for OS across all patients (Table 4), but favorable prognostic trends were noted for HER2 (HR=0.78, CI 0.60-1.02, p=0.071) and EGF (HR=0.84, CI 0.68-1.03, p=0.093). For OS, EREG expression was favorably prognostic for OS in the KRAS WT group (HR=0.87, CI 0.77-0.98, p=0.017). For PFS, HER2 (HR=0.64, CI 0.49-0.85, p=0.002) and EREG (HR=0.89, CI 0.80-0.98, p=0.016) were favorable prognostic markers across all patients. This effect seems to be driven by the KRAS-WT subgroup. Both HER2 (HR=0.66, CI 0.47-0.92, p=0.013) and EREG (HR=0.84, CI 0.74-0.96, p=0.008) were significant prognostic markers in the KRAS-WT group, but failed to show significance in the KRAS-Mut group (HER2 p=0.123, EREG p=0.526). The prognostic associations of each assayed gene with OS and PFS are included in FIGS. 2 and 3.

TABLE 4
Prognostic analyses of all markers for association with OS and PFS

| | All patients | | KRAS-WT | | KRAS-Mut | |
|---|---|---|---|---|---|---|
| Gene | HR (95% CI) | P | HR (95% CI) | P | HR (95% CI) | P |
| OS | | | | | | |
| AREG | 1.01 (0.88-1.15) | 0.923 | 0.97 (0.82-1.16) | 0.750 | 1.07 (0.89-1.30) | 0.475 |
| BTC | 1.01 (0.85-1.21) | 0.903 | 1.05 (0.83-1.34) | 0.678 | 1.01 (0.75-1.35) | 0.963 |
| CD73 | 1.05 (0.91-1.21) | 0.495 | 1.06 (0.88-1.27) | 0.536 | 1.04 (0.83-1.30) | 0.751 |
| DUSP4 | 0.99 (0.86-1.13) | 0.884 | 1.04 (0.89-1.23) | 0.599 | 0.91 (0.70-1.18) | 0.473 |
| EGF | 0.84 (0.68-1.03) | 0.093 | 0.81 (0.63-1.04) | 0.098 | 1.04 (0.61-1.76) | 0.890 |
| EGFR | 1.09 (0.91-1.30) | 0.372 | 1.04 (0.81-1.34) | 0.748 | 1.18 (0.88-1.59) | 0.272 |
| EPGN | 0.86 (0.60-1.23) | 0.399 | 0.96 (0.58-1.59) | 0.871 | 1.00 (0.59-1.68) | 0.988 |
| EREG | 0.94 (0.86-1.03) | 0.212 | 0.87 (0.77-0.98) | 0.017 | 1.07 (0.91-1.25) | 0.405 |
| HBEGF | 0.67 (0.73-1.04) | 0.121 | 0.86 (0.66-1.12) | 0.261 | 0.87 (0.68-1.11) | 0.250 |
| HER2 | 0.78 (0.60-1.02) | 0.071 | 0.83 (0.61-1.14) | 0.246 | 0.72 (0.41-1.28) | 0.264 |
| HER3 | 0.98 (0.81-1.18) | 0.831 | 1.03 (0.81-1.31) | 0.785 | 0.90 (0.64-1.28) | 0.565 |
| HER4 | 0.88 (0.70-1.11) | 0.283 | 0.87 (0.64-1.19) | 0.391 | 0.84 (0.53-1.31) | 0.414 |
| PHLDA1 | 1.06 (0.87-1.29) | 0.567 | 1.06 (0.81-1.38) | 0.679 | 1.21 (0.85-1.72) | 0.299 |
| TGFA | 1.01 (0.83-1.22) | 0.952 | 1.06 (0.85-1.32) | 0.621 | 0.84 (0.54-1.29) | 0.422 |
| PFS | | | | | | |
| AREG | 0.91 (0.80-1.03) | 0.144 | 0.90 (0.77-1.06) | 0.220 | 0.93 (0.77-1.13) | 0.461 |
| BTC | 0.89 (0.76-1.05) | 0.172 | 0.94 (0.75-1.17) | 0.578 | 0.84 (0.63-1.12) | 0.254 |
| CD73 | 0.99 (0.86-1.14) | 0.910 | 1.02 (0.55-1.22) | 0.855 | 0.97 (0.78-1.21) | 0.799 |
| DUSP4 | 0.95 (0.83-1.08) | 0.412 | 0.98 (0.84-1.15) | 0.799 | 0.89 (0.69-1.14) | 0.360 |
| EGF | 0.89 (0.74-1.07) | 0.223 | 0.86 (0.68-1.09) | 0.202 | 1.10 (0.75-1.80) | 0.492 |
| EGFR | 0.89 (0.73-1.07) | 0.220 | 0.80 (0.58-1.09) | 0.168 | 0.95 (0.73-1.23) | 0.696 |
| EPGN | 0.96 (0.67-1.36) | 0.815 | 1.04 (0.62-1.75) | 0.890 | 1.01 (0.60-1.71) | 0.963 |
| EREG | 0.89 (0.80-0.98) | 0.016 | 0.84 (0.74-0.96) | 0.008 | 0.95 (0.82-1.11) | 0.526 |
| HBEGF | 0.87 (0.73-1.03) | 0.117 | 0.92 (0.71-1.19) | 0.507 | 0.83 (0.66-1.04) | 0.103 |
| HER2 | 0.64 (0.49-0.85) | 0.002 | 0.66 (0.47-0.92) | 0.013 | 0.65 (0.38-1.12) | 0.123 |
| HER3 | 0.87 (0.74-1.04) | 0.127 | 0.91 (0.73-1.14) | 0.425 | 0.80 (0.59-1.10) | 0.174 |
| HER4 | 0.80 (0.62-1.02) | 0.067 | 0.79 (0.56-1.12) | 0.180 | 0.77 (0.50-1.17) | 0.180 |
| PHLDA1 | 0.95 (0.79-1.15) | 0.618 | 0.97 (0.76-1.24) | 0.827 | 1.00 (0.71-1.39) | 0.976 |
| TGFA | 0.90 (0.73-1.12) | 0.359 | 0.95 (0.74-1.23) | 0.704 | 0.81 (0.53-1.22) | 0.306 |

### Predictive Gene Expression Biomarkers

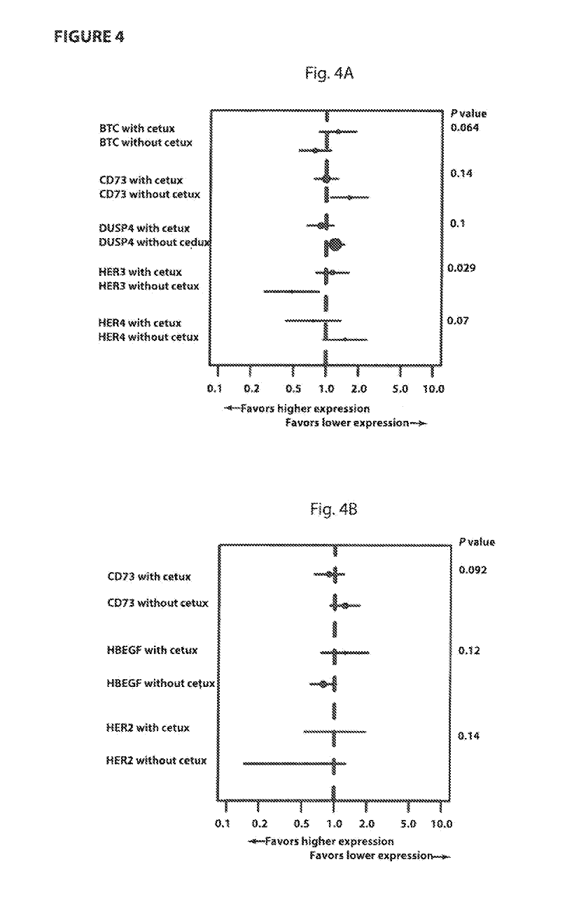

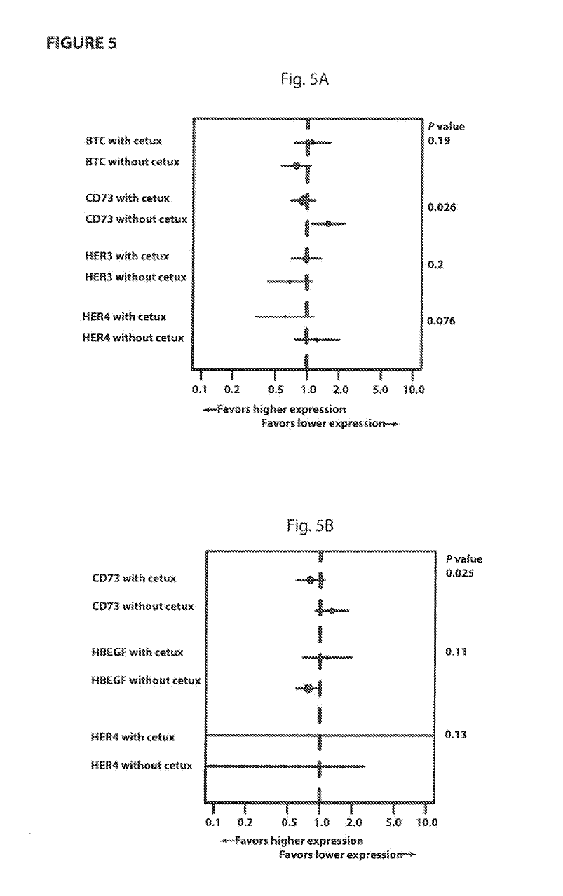

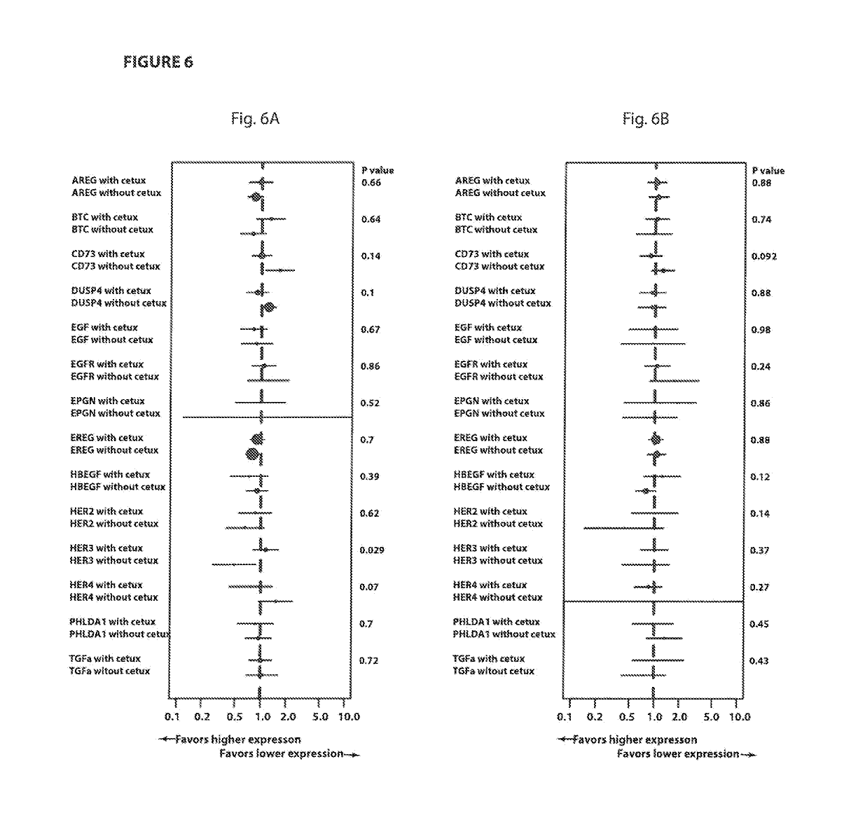

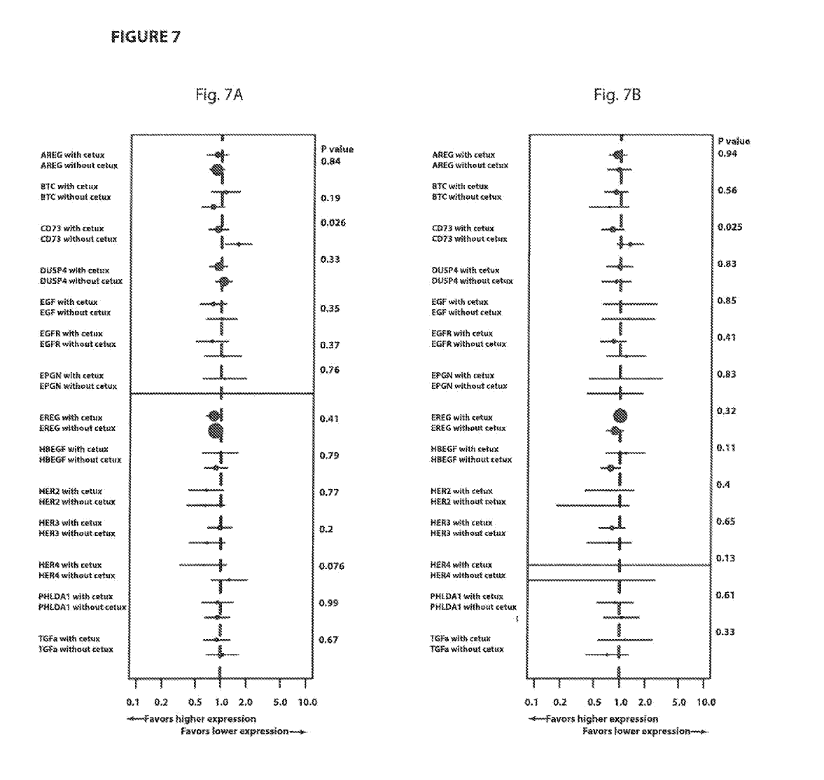

Cox proportional hazards models of OS and PFS were used to test for interaction between treatment and continuous tissue gene expression, and identified expression of HER3 and CD73 as potential predictive markers for benefit or lack of benefit from cetuximab. Forest plots of the hazard ratio of gene expression by treatment group are presented for OS and PFS outcomes. Markers with an interaction p-value≦0.2 are shown in FIGS. 4 and 5, while a complete analysis showing all markers is included in FIGS. 6 and 7.

Higher levels of HER3 expression showed evidence of being predictive for lack of benefit from cetuximab, an effect that appeared restricted to the KRAS-WT group. For OS in the KRAS-WT group, the HR for chemo+cetuximab was 1.15 (CI 0.81-1.62) and the HR in the chemo only group was 0.48 (CI 0.27-0.87; interaction p=0.029) (FIG. 4A). However, in the KRAS-Mut population, HER3 was not predictive of either OS or PFS benefit from cetuximab (FIGS. 4B and 5B).

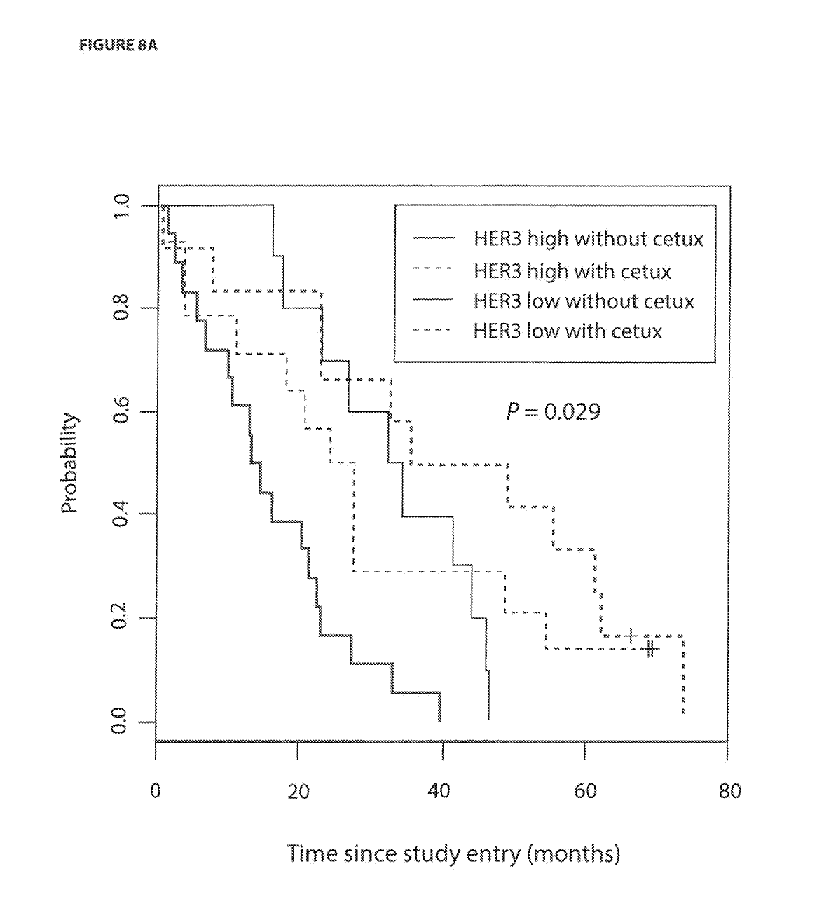

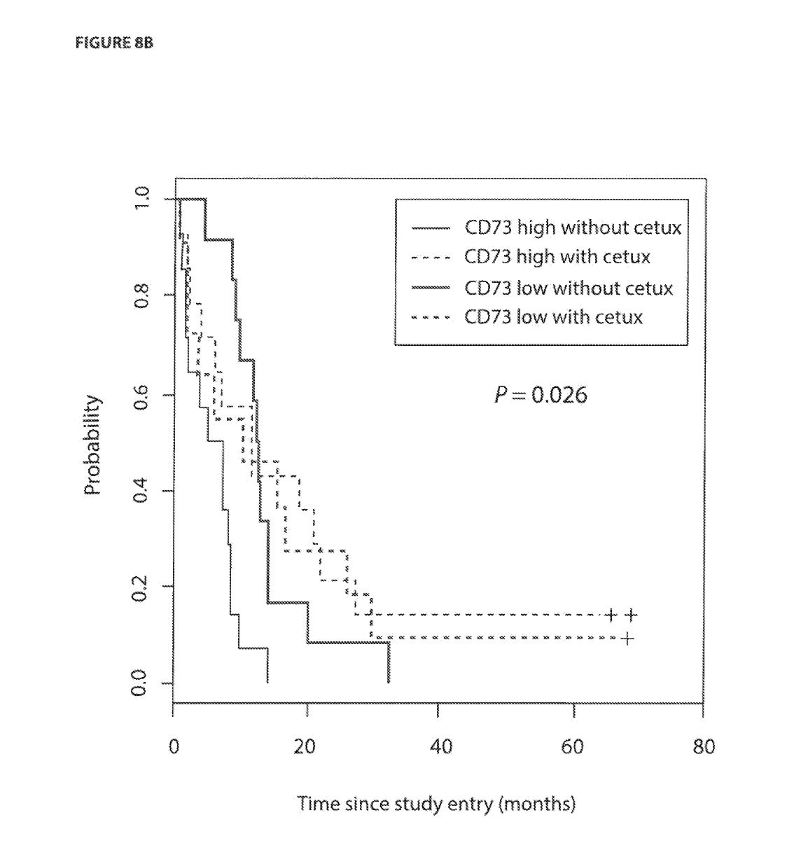

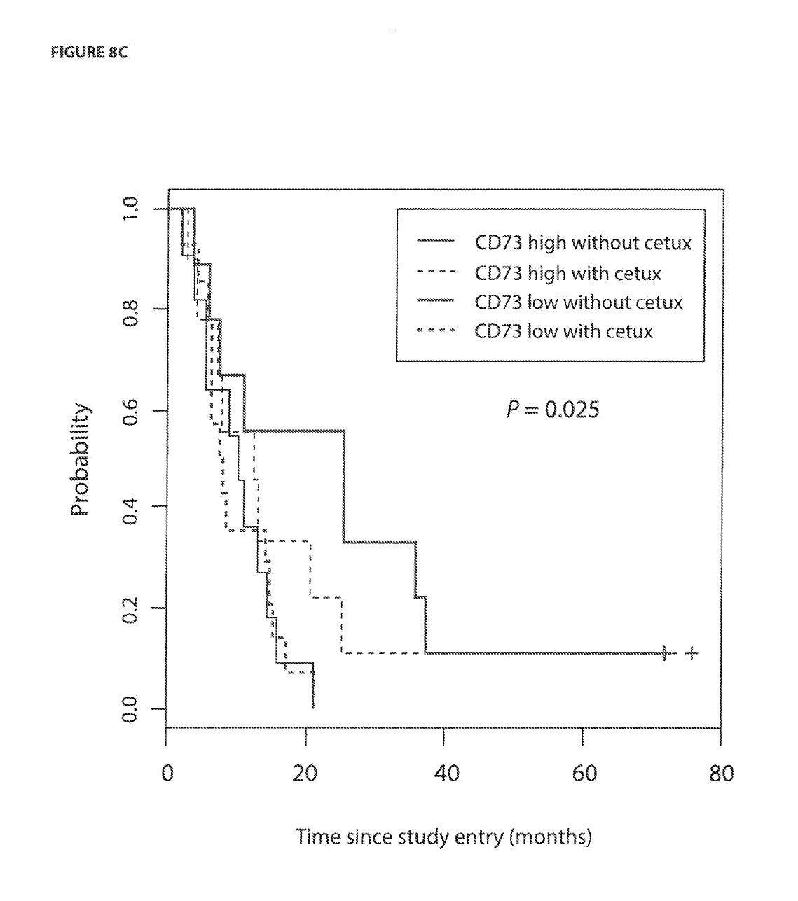

Gene expression of CD73 showed a similar trend toward predicting for OS benefit from cetuximab in the KRAS-WT (interaction p=0.14) and KRAS-Mut (interaction p=0.092) groups. Higher levels of CD73 expression predict for PFS benefit from cetuximab, an effect that appeared to be consistent in both KRAS-WT and KRAS-Mut groups. For PFS in the KRAS-WT group, the HR was 0.91 (CI 0.70-1.18) for the chemo+cetuximab group and 1.57 (CI 1.11-2.23) for the chemo only group (interaction p=0.026). For PFS in the KRAS-Mut group, the HR was 0.80 (CI 0.60-1.07) for the chemocetuximab and 1.29 (CI 0.91-1.83) for the chemo only group (interaction p=0.025). Kaplan-Meier plots of high and low expression of HER3 and CD73 (dichotomized at the median) are also shown (FIG. 8).

### RNA Expression Results for CALGB 80203 Clinical Samples

Our analysis of CALGB 80203 is one the largest analyses of gene expression in a first-line mCRC study to date. A key advantage of CALGB 80203 for biomarker analyses is its use of randomization between chemotherapy with and without cetuximab. Without randomization, the prognostic and predictive roles of candidate markers cannot be distinguished and their roles may be confounded or obscured.

Our findings suggest both the HER axis and inflammatory pathways in mediating resistance to cetuximab. High HER3 levels were associated with both resistance and lack of benefit from cetuximab treatment. This effect was most prominent in patients whose tumors were KRAS-WT. High HER3 expression predicts for a lack of benefit in OS with cetuximab treatment. The same trend holds in PFS, but it does not reach the level of statistical significance in this analysis. HER3 is a member of the same receptor family as EGFR, but unlike EGFR it has no intrinsic tyrosine-kinase activity. HER3 is capable of forming heterodimers with members of the EGFR receptor family and it may play a role in the development of resistance to EGFR-targeting therapies. Expression of other markers in the HER axis showed a trend for predicting benefit from cetuximab.

We also identified tissue (CD73 expression as a potential predictive marker for benefit from cetuximab. Low CD73 expression in tumor tissue is predictive for lack of benefit in both OS and PFS with cetuximab treatment. Surprisingly, our results were consistent in both KRAS-WT and KRAS-Mut populations. CD73 is a membrane-associated nucleotidase localized on the exterior surfaces of cells. It plays a central role in the dephosphorylation of extracellular ATP to adenosine. The enzymatic activity of CD73 in this process is important for the regulation of inflammatory and immune systems.

In conclusion, using samples from the randomized CALGB 80203 study in first-line mCRC we identified potential candidate predictors of benefit from cetuximab, including HER3 and CD73. These data implicate specific and targetable factors in the HER axis and inflammation as key mediators of resistance to cetuximab.

### Example 2

### Blood-based Markers of Efficacy to Cetuximab Treatment in Metastatic Colorectal Cancer

### Patients and Methods

### Study Design and Patients

Design details of the CALGB 80203 study are described above in Example 1. Patients with previously untreated, advanced or metastatic adenocarcinoma of the colon or rectum were assigned to FOLFIRI, FOLFIRI plus cetuximab, FOLFOX, or FOLFOX plus cetuximab treatment groups. This was a multi-center trial approved by the institutional review boards at each participating institution, and all patients gave written informed consent before enrollment.

### Sample Collection

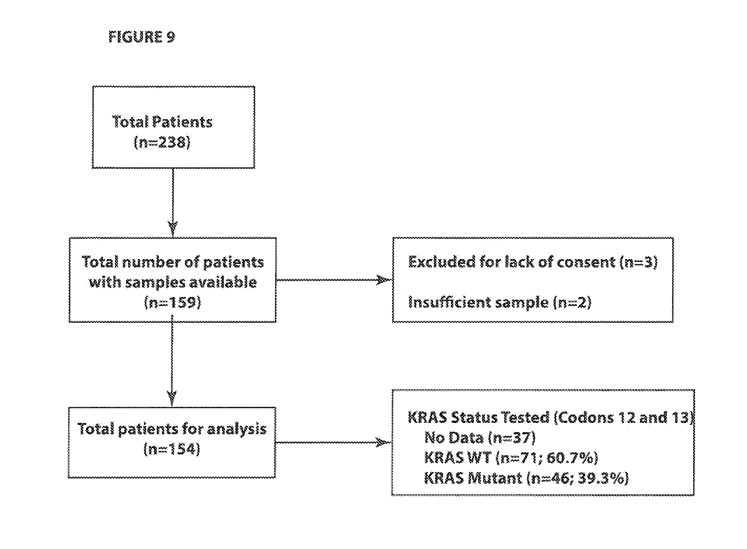

Plasma from 154 patients was collected and the amount of soluble EGFR related proteins in their plasma was directly interrogated using ELISA-based techniques (FIG. 9). Characteristics of the 154 patients tested are shown in Table 5. Peripheral venous blood was collected at baseline from consenting patients into lavender (EDTA anticoagulant) vacutainers. Samples were centrifuged at 2500×g for 15 minutes within 30 minutes of collection. Plasma was aliquoted into cryovials, frozen in liquid nitrogen, and samples were shipped for centralized storage at the CALGB (now part of the Alliance for Clinical Trials in Oncology) Pathology Coordinating Office. Before analysis, all patient samples were shipped to our laboratory (Duke/Alliance Molecular Reference Lab), thawed on ice, re-aliquoted based on specific assay requirements and stored at −80° C.

TABLE 5
PATIENT CHARACTERISTICS
Patient Characteristics

| | Chemo | Chemo + C | Total | |
|---|---|---|---|---|
| | (N = 76) | (N = 78) | (N = 154) | p-value |
| Age | Number (% Total) | Number (% Total) | Number (% Total) | 0.30 |

| | | | |
|---|---|---|---|
| 20-29 | 2 (16%) | 1 (13%) | 3 (1.9%) |
| 30-39 | 5 (6.6%) | 3 (3.8%) | 8 (5.2%) |
| 40-49 | 7 (9.2%) | 13 (16.7%) | 20 (13.0%) |
| 50-59 | 20 (26.3%) | 15 (19.2%) | 35 (22.7%) |
| 60-69 | 29 (38.2%) | 24 (30.8%) | 53 (34.4%) |
| 70+30 | 13 (17.1%) | 22 (282%) | 35 (22.7%) |

| Gender | | | | | 0.77 |
|---|---|---|---|---|---|
| | Male | 47 (61.8%) | 50 (64.1%) | 97 (63.0%) | |
| | Female | 29 (38.2%) | 28 (35.9%) | 57 (37.0%) | |
| Race | | | | | 0.23 |
| | White | 64 (84.2%) | 71 (91.0%) | 135 (87.7%) | |
| ECOG PS | | | | | 0.20 |
| | 0 | 34 (44.7%) | 43 (55.1%) | 77 (50.0%) | |
| | 1 | 42 (55.3%) | 35 (44.9%) | 77 (50.0%) | |
| KRAS Status | | | | | 0.99 |
| | Missing | 20 | 17 | 37 | |
| | KRAS Mut | 22 (39.3%) | 24 (39.3%) | 46 (39.3%) | |
| | KRAS WT | 34 (60.7%) | 37 (60.7%) | 71 (60.7%) | |

### KRAS Mutational Analysis

KRAS mutation status was determined using the TheraScreen KRAS Mutation Test Kit (Qiagen, Manchester, UK 870021), which is able to detect the seven common mutations of the KRAS gene at codons 12 and 13. Analysis was performed in the CALGB/Alliance molecular reference laboratory of Dr. Greg Tsongalis at Dartmouth Medical School.

### Plasma Protein Analysis

We measured six markers in the plasma. The markers include EGF, HB-EGF, sEGFR, sHER2, sHER3, CD73. EGF, HBEGF, sEGFR, and sHER2 were analyzed using the Searchlight platform (Aushon Biosystems, Inc., Billerica, Mass.) following the manufacturer's protocol. Plasma samples were thawed on ice, centrifuged at 20,000×g for 5 minutes to remove precipitate and loaded onto SearchLight plates with recombinant protein standards. Samples and standards were incubated at room temperature for 1 hour shaking at 950 rpm (Lab-Line Titer Plate Shaker, Model 4625, Barnstead, Dubuque, Wis.). Plates were washed three times using a plate washer (Biotek Instruments, Inc., Model ELx405, Winooski, Vt.), biotinylated secondary antibody was added, and plates were incubated for 30 min. After washes, streptavidin-HRP was added, incubated for 30 min, plates were washed again, and SuperSignal substrate was added. Images were taken within 10 minutes and subsequently analyzed using SearchLight array analyst software.

sHER3 and CD73 were analyzed using novel assays on the Meso Scale Discovery ELISA platform. For sHER3, ELISA plates were coated overnight with 4 μg/ml HER3 capture antibody (R&D Systems, Minneapolis, Minn. MAB3481). After sample incubation, HER3 was detected using 1 μg/ml biotinylated HER3 antibody (R&D Systems, Minneapolis, Minn. BAF234) and 5 μg/ml streptavidin-conjugated SulfoTag (Meso Scale Discovery, Rockville, Md. R32AD-5). For CD73, ELISA plates were coated overnight with 3.3 μg/ml CD73 capture antibody (BD Biosciences, San Jose, Calif. 550256). After sample incubation, CD73 was detected using 1 μg/ml antibody (Invitrogen/Life Technologies, Grand Island, N.Y. 41-0200) conjugated to MSD SulfoTag according to the manufacturer's instructions (Meso Scale Discovery, Rockville, Md. R91AN-1). Samples were quantified using MSD Discovery Workbench software (Meso Scale Discovery, Rockville, Md.). All assays were performed in duplicate and laboratory personnel were blinded to clinical outcome.

### Statistical Analysis

Univariate Cox regression was used to identify markers prognostic of the primary outcome, overall survival (OS), and secondary outcome (PFS). The resulting p-values, hazard ratios, and 95% confidence intervals were calculated. The resulting effect sizes were visualized in the form of forest plots. To identify predictive markers, expression level was correlated with clinical outcomes (OS and PFS) using multiplicative Cox proportional hazards models to test for interaction between marker expression and treatment (chemo vs. chemo+cetuximab). Kaplan-Meier plots of OS and PFS were generated for predictive markers, with separate curves for each combination of treatment group and expression level (where expression level is dichotomized at the median as “high” or “low”). Analyses were conducted using all patients, as well as separately within KRAS-WT and KRAS-Mut subgroups, due to known differential responses to cetuximab across these populations. Marker levels were log-transformed and analyzed as continuous values.

### Results

### Patient Characteristics

Plasma samples were available for biomarker analysis from 154 of the 238 patients enrolled. The characteristics of this biomarker population reflected the characteristics of the overall study population (Table 5). As previously reported, there were no observed differences in outcomes between the groups that received FOLFOX or FOLFIRI chemotherapy, so these groups were combined into chemotherapy (chemo) alone (FOLFOX or FOLFIRI) and chemo+cetuximab groups for this study. No significant differences in the characteristics of the chemo and chemo+cetuximab groups were observed. KRAS mutational analysis was limited to the seven common mutations of the KRAS gene at codons 12 and 13. Extended RAS mutational analyses were not performed. KRAS mutational status was only available for 117 (76%) of the patients in this group. In the blood-based biomarker cohort the rate of KRAS mutation is 39.3%, slightly less than the rate of 43.0% in the parent study and 46.6% in our previous analysis of mRNA expression from FFPE samples (See Example 1). A CONSORT diagram is presented in FIG. 9.

### Biomarker Analysis

The six markers of interest were chosen based on their direct role in EGFR signaling, previous examination of mRNA levels in archived FFPE tumor samples, and the ability to assess each soluble marker in patient plasma. The levels of EGFR markers in blood were measured and associated with both the primary (overall survival, OS) and secondary (progression-free survival, PFS) outcomes. The characteristics of the assayed markers are shown in Table 6. The EGFR ligands (EGF, HBEGF) were present at lower levels, but were observed to have higher levels of variability between patients. Baseline levels of the EGF and HBEGF ligands were correlated (Spearman correlation coefficient ρ=0.48), as were levels of sHER2 and sHER3 (ρ=0.45). No other marker pairs showed strong correlations (ρ<0.3) (Table 7). Prognostic analyses were performed using baseline data from all available patients independent of treatment arm, and predictive analyses were performed using a Cox proportional hazard model with continuous values for the protein analytes. To further assess the role that KRAS mutational status has on subsequent biomarker determinations, separate analyses were performed for patients with KRAS-WT and KRAS-Mut tumors to account for the role of KRAS mutational status plays in cetuximab sensitivity and resistance. There were no associations observed for any marker tested and KRAS mutation status.

TABLE 6
MARKER PROPERTIES

| | N | Units | Average | Median | Range |
|---|---|---|---|---|---|
| EGF | 154 | pg/ml | 37.1 | 19.8 | 0.3-361.3 |
| HBEGF | 154 | pg/ml | 18.3 | 14.8 | 5.6-2352 |
| EGFR | 154 | ng/ml | 25.9 | 25.6 | 3.5-49.3 |
| sHER2 | 154 | ng/ml | 3.7 | 3.2 | 1.4-25.1 |
| sHER3 | 146 | ng/ml | 11.6 | 11.0 | 6.6-45.8 |
| CD73 | 137 | ng/ml | 8.5 | 4.3 | 0.7-67.4 |

TABLE 7
SPEARMANN CORRELATION COEFFICIENTS
FOR EACH MARKER ANALYZED

| | EGF | HBEGF | EGFR | sHER2 | sHER3 |
|---|---|---|---|---|---|
| EGF | 1 | 0.48 | −0.09 | 0.08 | 0.29 |
| HBEGF | | | −0.06 | 0.08 | 0.11 |
| EGFR | | | | 0.22 | 0.08 |
| sHER2 | | | | 1 | 0.45 |
| sHER3 | | | | | 1 |

### Ligand Markers

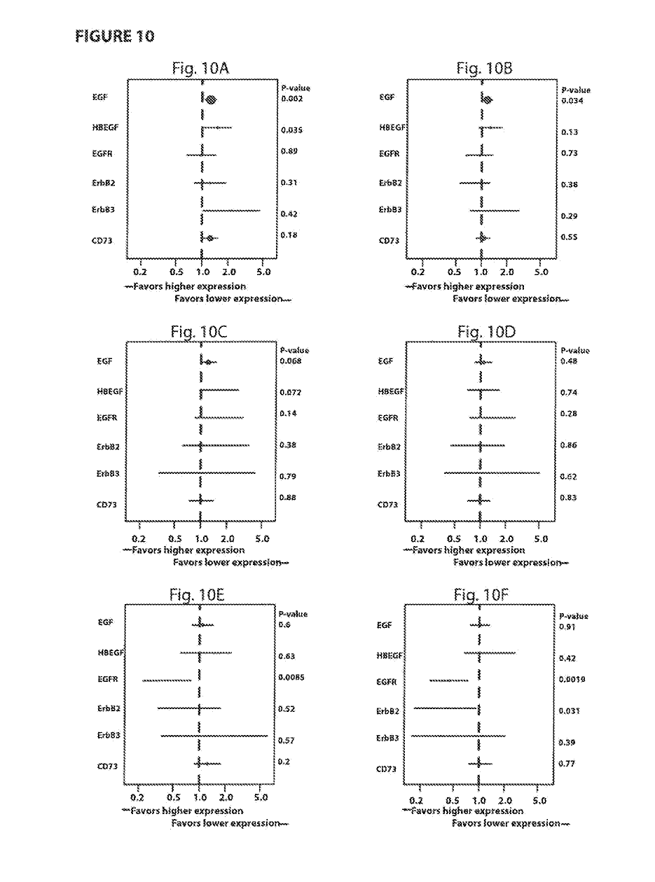

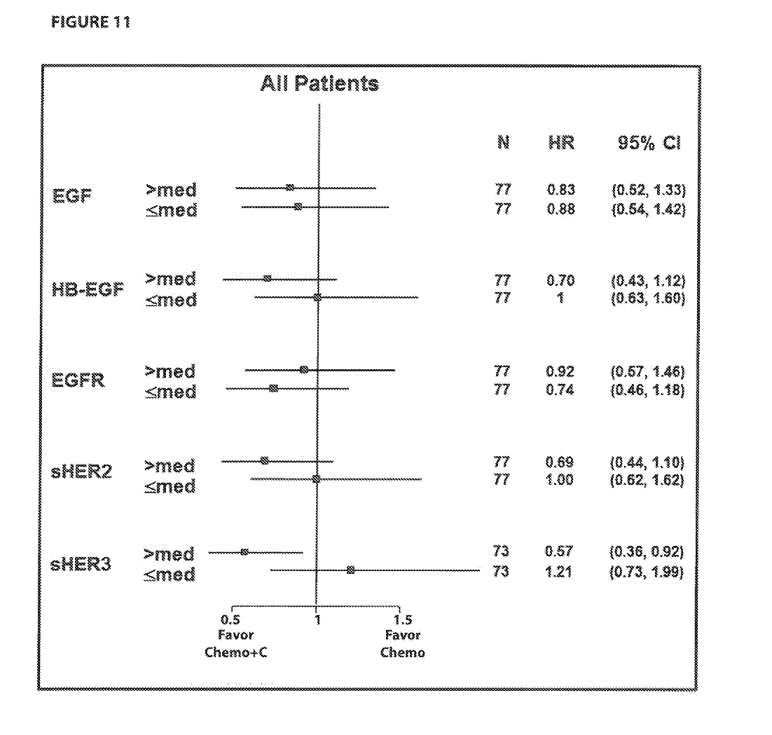

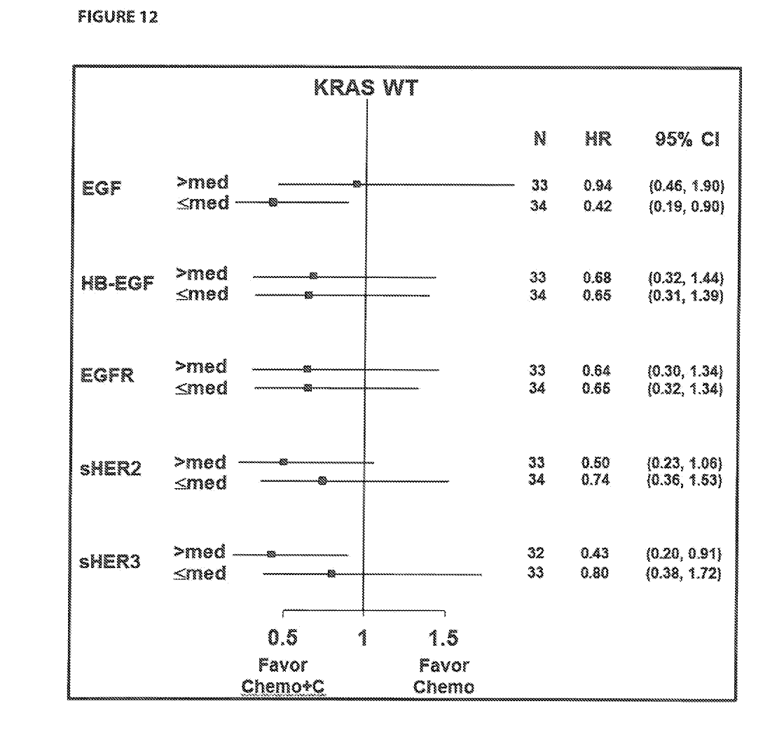

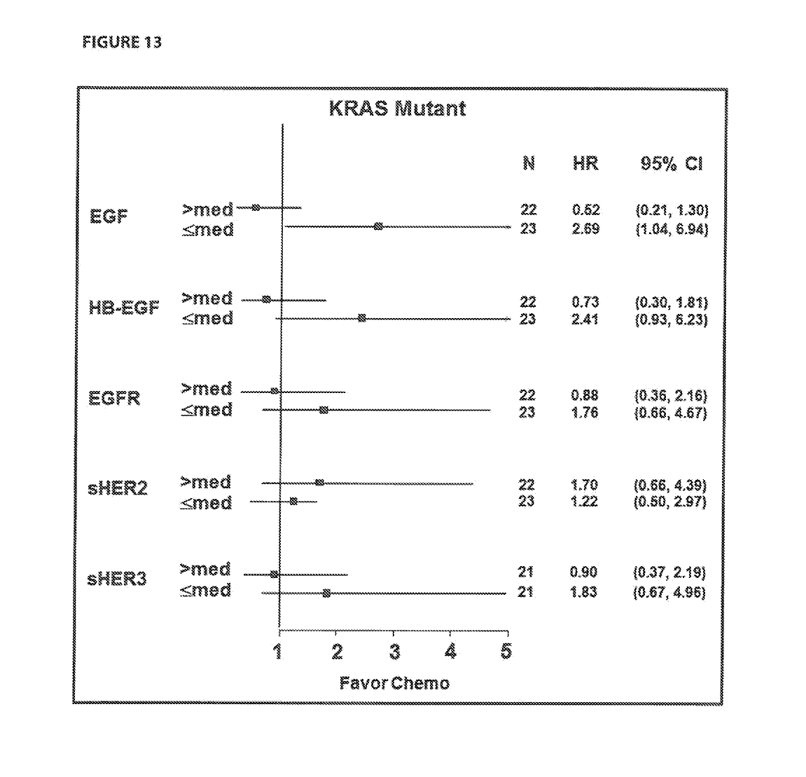

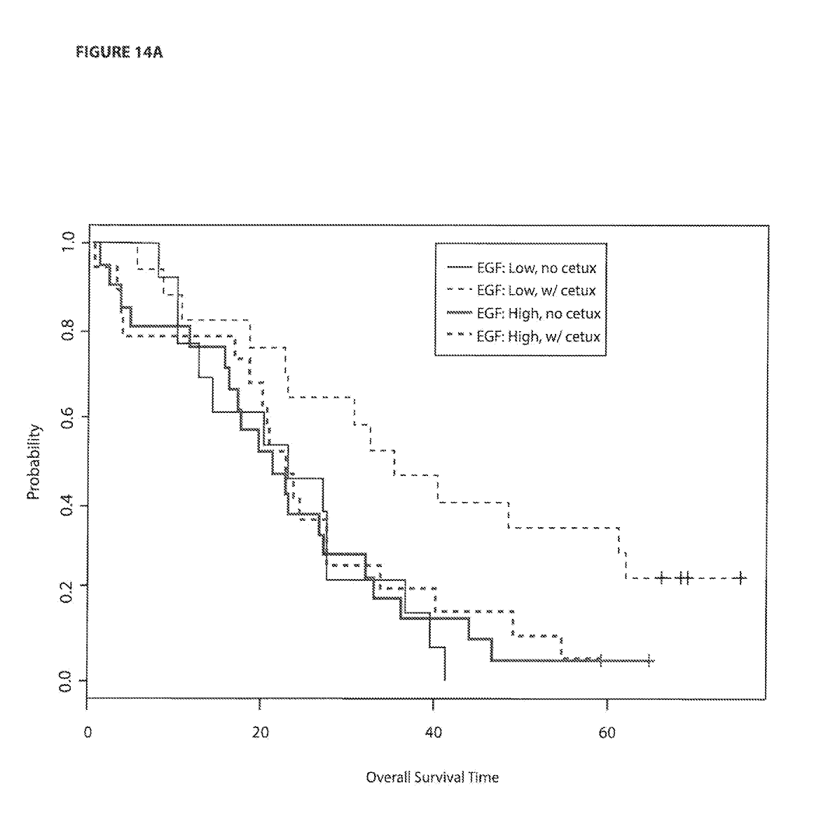

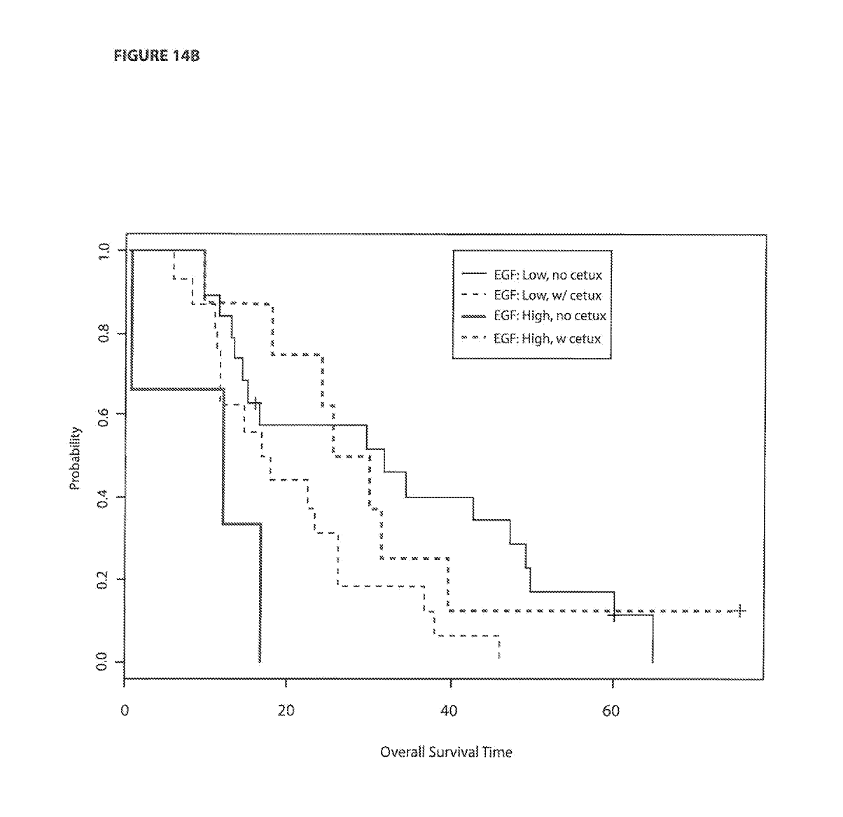

EGF protein levels were prognostic for OS (HR=1.25, 95% CI 1.09-1.45, p=0.002) and PFS (HR=1.17, 95% CI 1.01-1.34, p=0.035) across all patients, independent of treatment arm or KRAS mutation status (FIGS. 10A and B and FIG. 11). This effect was not observed in the KRAS subgroups (FIG. 10C-D and FIG. 12 KRAS wild-type and FIG. 10 E-F and FIG. 13 for KRAS mutant). EGF showed a trend towards being prognostic for OS in KRAS-WT patients (HR=1.21, 95% CI 0.99-1.49, p=0.068), but showed no association with PFS in this subgroup (p=0.482). Furthermore, EGF was not associated with either OS (p=0.596) or PFS (p=0.913) in KRAS-Mut patients.

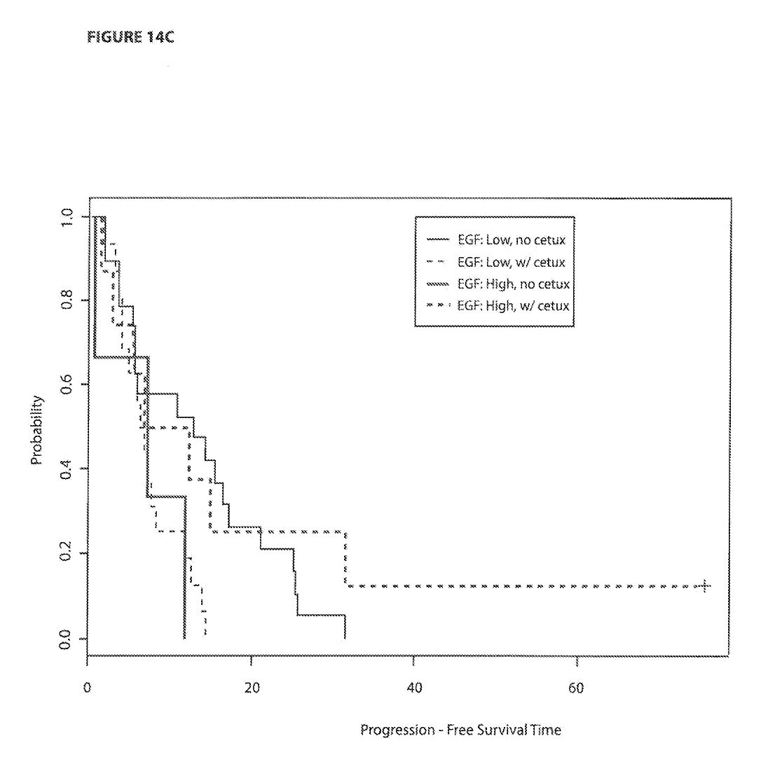

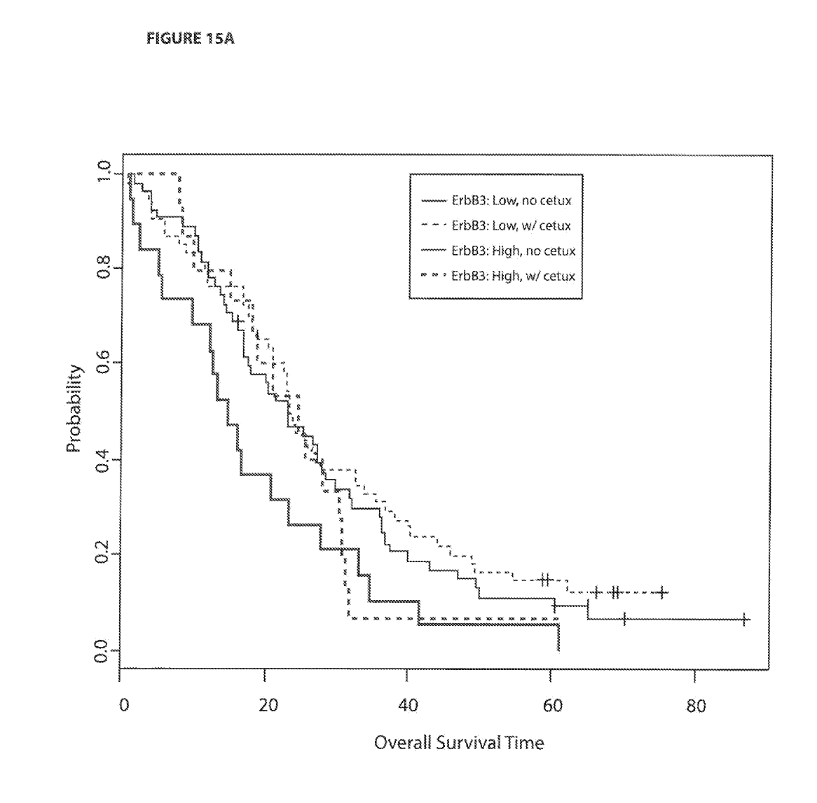

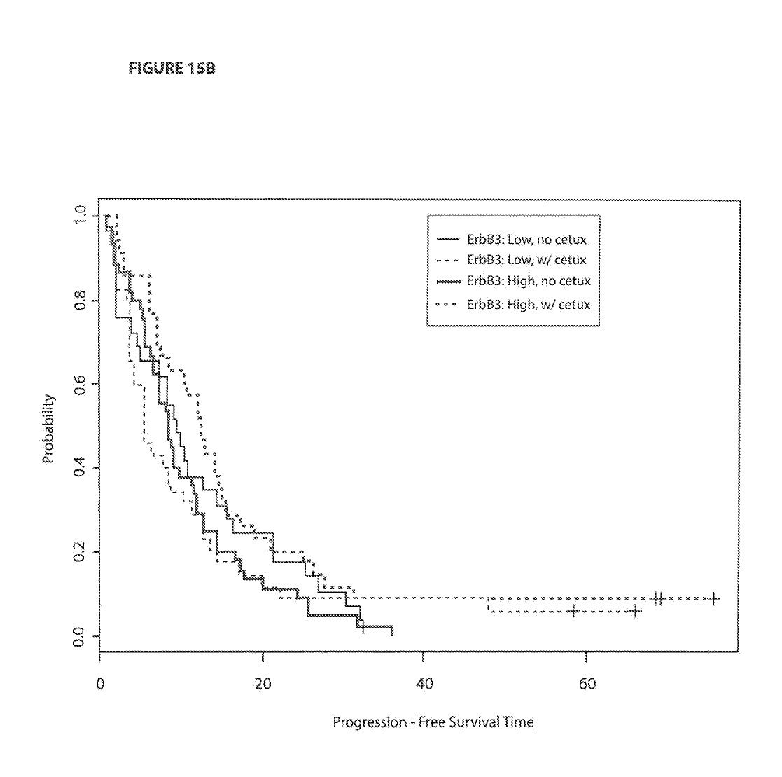

EGF protein levels were not predictive of OS (interaction p=0.748) or PFS (interaction p=0.233) benefit from cetuximab across all patients, but EGF levels were predictive within the individual KRAS subgroups. In KRAS-WT patients, higher EGF levels were predictive of lack of OS benefit from cetuximab (Chemo HR=0.98, 95% CI 0.74-1.29; Chemo+cetux HR=1.54, 95% CI 1.05-2.25; interaction p=0.045) (FIG. 14A), but were not predictive of PFS (interaction p=0.719). Reciprocally, high EGF was predictive of benefit in OS (Chemo HR=1.72, 95% CI 1.02-2.92; Chemo+cetux HR=0.90, 95% CI 0.67-1.21; interaction p=0.026) and PFS (Chemo HR=2.16 95% CI 1.2.9-3.63; Chemo+cetux HR=0.76 95% CI 0.56-1.03; interaction p=0.001) from cetuximab in KRAS-Mut patients (FIGS. 14B and C), though this was primarily due to EGF being associated with increased risk in the control group.

Levels of HBEGF were prognostic for OS across all patients (HR=1.49, 95% CI 1.03-2.16, p=0.035) and showed a trend towards being prognostic KRAS-WT patients (HR=1.61 95% CI 0.96-2.69, p=0.072). HBEGF levels were not significantly predictive for either survival endpoint across all patients or in either KRAS subgroup.

### Receptor and Immune Markers

EGFR is the direct molecular target of cetuximab and levels of EGFR protein have been studied extensively as a potential predictive biomarker of cetuximab efficacy. In this study, plasma levels of sEGFR were not prognostic for OS or PFS across all patients or in the KRAS-WT subgroup. However, sEGFR levels were prognostic for both OS (HR=0.43, 95% CI 0.23-0.80, p=0.009) and PFS (HR=0.44 95% CI 0.26-0.74, p=0.002) specifically in KRAS-Mut patients. Plasma sEGFR showed a slight trend toward predicting OS benefit from cetuximab in KRAS-Mut patients (Chemo HR=1.21, 95% CI 0.27-5.38; Chemo+cetuximab HR=0.33, 95% CI 0.16-0.67; interaction p=0.210). This effect was not observed for PFS (interaction p=0.997).

Plasma levels of sHER2 protein were not generally associated with survival endpoints in this study though they were prognostic in KRAS-Mut patients (HR=0.40, 95% CI 0.17-0.92, p=0.031). Levels of sHER3 were prognostic for OS (HR=2.17, 95% CI 1.03-4.58, p=0.042) across all patients, but not for the KRAS-WT and KRAS-Mut subgroups. Levels of sHER3 were predictive for both OS (Chemo HR=4.82, 95% CI 1.68-13.84; Chemo+cetuximab HR=0.95, 95% CI 0.31-2.95; interaction p=0.046) (FIG. 15A) and PFS (Chemo HR=3.90, 95% CI 1.41-10.80; Chemo+cetuximab HR=0.66, 95% CI 0.25-1.78; interaction p=0.032) across all patients (FIG. 15B). It should be noted that the predictive ability of sHER3 was sensitive to the presence of an outlier with a high level of plasma sHER3. When this patient was removed from the analysis sHER3 was no longer predictive at p=0.05, but the trends remained (OS interaction p=0.128, PFS interaction p=0.098). This outlier had the third shortest OS time in this study and did not have extreme values for any of the other markers examined, possibly indicating that the high sHER3 levels were biologically relevant and not an artifact of sample handling.

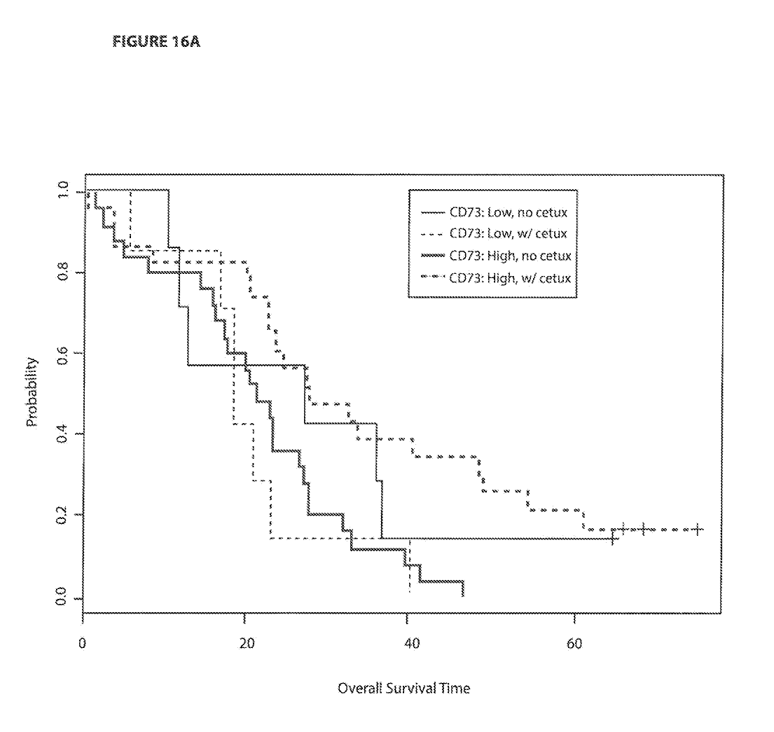

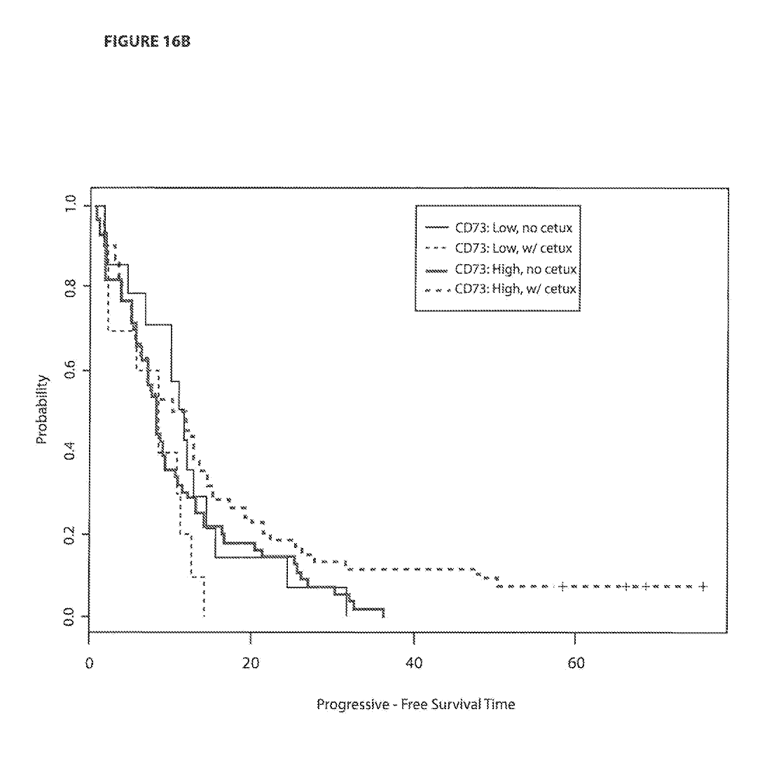

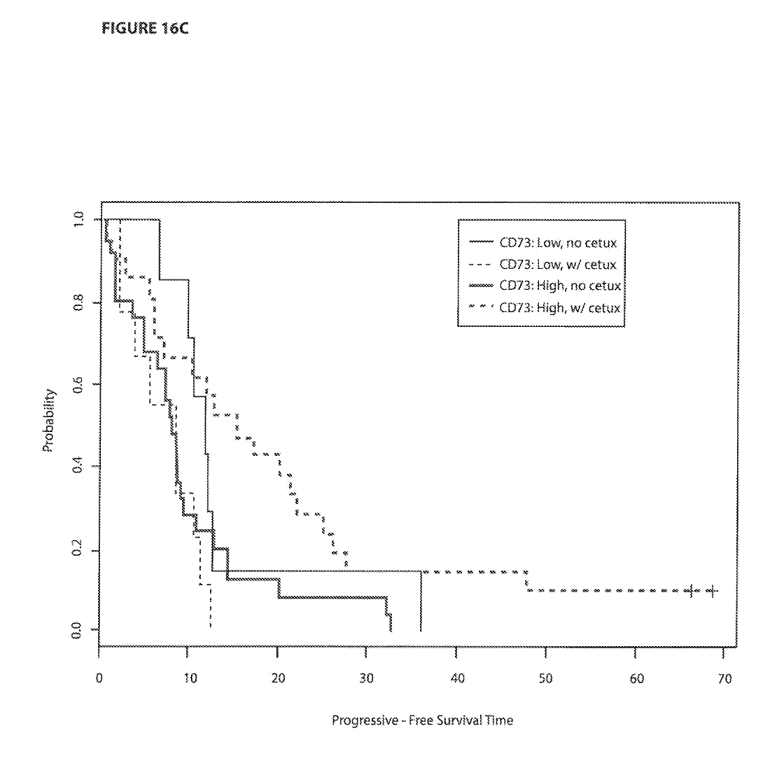

As an immune-modulatory, extracellular AMP 5′-nucleotidase CD73 is not known to influence the EGFR pathway in the same direct manner as the other markers examined here, but the predictive nature of CD73 tumor mRNA expression in this trial population justified examination of the plasma protein in this analysis. Plasma CD73 was prognostic for OS across all patients (HR=1.26, 95% CI 1.04-1.52, p=0.018). CD73 protein levels showed a slight trend for predicting OS benefit from cetuximab across all patients (Chemo HR=1.41, 95% CI 1.10-1.80; Chemo+cetuximab HR=1.09, 95% CI 0.81-1.47; interaction p==0.204) and were predictive of OS benefit in KRAS-WT patients (Chemo HR=1.28, 95% CI 0.88-1.84; Chemo+cetuximab HR=0.60, 95% CI 0.32-1.13; interaction p=0.049) (FIG. 16A). CD73 levels were predictive of PFS benefit across all patients (Chemo HR=1.38; 95% CI 1.08-1.77; Chemo+cetuximab HR=0.84, 95% CI 0.63-1.12; interaction p=0.018) (FIG. 16B) and in KRAS-WT patients (Chemo HR=1.32, 95% CI 0.92-1.90; Chemo+cetuximab HR=0.61, 95% CI 0.36-1.04; interaction p=0.017) (FIG. 16C). No predictive effects were observed in KRAS-Mut patients.

Comparison of Plasma Proteins and Tumor mRNA Expression

In Example 1, we identified several potential prognostic and predictive biomarkers from CALGB 80203 evaluating mRNA expression from FFPE tumor biopsies. In that work, we found that tumor expression of HER3 and CD73 were predictive biomarkers for cetuximab. The concordance between tumor-based gene expression and plasma-derived protein levels were evaluated. There were 71 patients who had both FFPE and plasma sample available for this concordance analysis. For most markers in these 71 patients there was little association between tumor mRNA expression and plasma protein levels. EGF, HBEGF, EGFR, HER2 and CD73 exhibited no correlation between plasma protein levels and tumor mRNA expression levels. However, plasma sHER3 protein and tumor HER3 mRNA expression were correlated with one another (r=0.22, p=0.010). Further investigation is required to confirm whether plasma sHER3 is generally associated with tumor gene expression levels, or whether this was a coincidence of the current study population and whether the sHER3 measured in these patients may be tumor-derived. Plasma levels of sHER3 protein identified patients in the control arm with shorter OS in this study. This is in contrast with our studies examining tumor mRNA expression in this trial that found HER3 mRNA expression in the tumor identifies patients in the control arm with longer OS. In both cases the HR in the chemo+cetuximab arm was approximately 1, indicating that HER3 tumor mRNA expression and soluble plasma protein perhaps reflect an interaction with chemotherapy that is modulated by treatment with cetuximab.

TABLE 8
Univariate Overall Survival Prognostic Markers
at baseline for FOLFIRI/FOLFOX treatments
Below is a list of analytes that is prognostic univariately for overall
survival using the Cox Proportional Hazard model. On the right hand
side, it indicates the median survival time and its 95% CI for less than
median and greater than median level of the analytes. (p < .01)

| | | <=median | | >median | | <=med vs >med | |
|---|---|---|---|---|---|---|---|
| | | Median | | Median | | Hazard | |
| | p-value* | Survival | 95% CI | Survival | 95% CI | ratio | 95% CI |
| egf | 0.01815 | 27.6 | (23, 36.6) | 16.1 | (13, 22.8) | 1.75 | (1.09, 2.81) |
| erbb3 | 0.00017 | 27.6 | (16.7, 36.3) | 17.6 | (13.3, 23.1) | 1.73 | (1.07, 2.82) |

Note:
hazard ratio = [hazard_{>med}/hazard_{<med}]
*from Cox proportional hazard model using continuous analyte values.

TABLE 9
Univariate Overall Survival Prognostic Markers at baseline
for FOLFIRI/FOLFOX + C225 treatments KRAS WT
Below is a list of analytes that is prognostic univariately for overall
survival using the Cox Proportional Hazard model. On the right hand
side, it indicates the median survival time and its 95% CI for less than
median and greater than median level of the analytes. (p < .01)

| | | <=median | | >median | | <=med vs >med | |
|---|---|---|---|---|---|---|---|
| | | Median | | Median | | Hazard | |
| | p-value* | Survival | 95% CI | Survival | 95% CI | ratio | 95% CI |
| egf | 0.00529 | 32.6 | (23.0, 62.1) | 21.8 | (16.7, 40.0) | 2.00 | (0.96, 4.19) |

TABLE 10
Univariate Overall Survival Prognostic Markers at baseline
for FOLFIRI/FOLFOX + C225 treatments KRAS Mutant
Below is a list of analytes that is prognostic univariately for overall
survival using the Cox Proportional Hazard model. On the right hand
side, it indicates the median survival time and its 95% CI for less than
median and greater than median level of the analytes. (p < .01)

| | | <=median | | >median | | <=med vs >med | |
|---|---|---|---|---|---|---|---|
| | | Median | | Median | | Hazard | |
| | p-value* | Survival | 95% CI | Survival | 95% CI | ratio | 95% CI |
| egfr | 0.00190 | 17.8 | (11.7, 26.1) | 31.5 | (25.4, ∞) | 6.29 | (0.11, 0.78) |

TABLE 11
Univariate Overall Survival Predictive Markers at baseline
for FOLFIRI/FOLFOX vs FOLFIRI/FOLFOX + C225
Below is a list of analytes that is predictive univariately for overall survival using the
Cox Proportional Hazard model with significant Treatment and Analyte interaction term.
On the right hand side, it indicates the median survival time and its 95% CI for the
FOLFIRI/FOLFOX arm and the FOLFIRI/FOLFOX + C225 arm using the specified cutoff.

| | Treatment × | | | | FOLFIRI/FOLFOX | | F/F + C225 | |
|---|---|---|---|---|---|---|---|---|
| | Analyte | | | | Median | | Median | |
| Analyte | p-value | Cutoff | HR | 95% CI | Survival | 95% CI | Survival | 95% CI |
| | Median | | | | | | | |
| erbb3 | 0.0355 | >median | 0.57 | (0.36, 0.92) | 17.6 | (13.3, 23.1) | 28.8 | (25.1, 33.6) |
| | | 0.021 | | | | | | |
| | perm | | | | | | | |
| | 0.026 | | | | | | | |
| | continuous | | | | | | | |
| erbb3 | 0.032 | | | | | | | |

hazard ratio = [hazard_{F/F+C225}/hazard_{F/F}]

TABLE 12
Univariate Overall Survival Predictive Markers at baseline
for FOLFIRI/FOLFOX vs FOLFIRI/FOLFOX + C225 KRAS WT
Below is a list of analytes that is predictive univariately for overall survival using the
Cox Proportional Hazard model with significant Treatment and Analyte interaction term.
On the right hand side, it indicates the median survival time and its 95% CI for the
FOLFIRI/FOLFOX arm and the FOLFIRI/FOLFOX + C225 arm using the specified cutoff.

| | Treatment × | | | | FOLFIRI/FOLFOX | | F/F + C225 | |
|---|---|---|---|---|---|---|---|---|
| | Analyte | | | | Median | | Median | |
| Analyte | p-value | Cutoff | HR | 95% CI | Survival | 95% CI | Survival | 95% CI |
| | Median | | | | | | | |
| egf | 0.147 | <median | 0.39 | (0.18, 0.87) | 23.0 | (12.7, 39.6) | 33.9 | (22.6, 62.1) |
| | | 0.021* | | | | | | |
| | continuous | | | | | | | |
| egf | 0.008 | | | | | | | |
| | Median | | | | | | | |
| erbb3 | 0.573 | >median | 0.43 | (0.20, 0.91) | 20.7 | (15.8, 27.5) | 27.6 | (24.4, 54.5) |
| | | 0.028* | | | | | | |

*corresponds to the hazard

TABLE 13
Univariate Overall Survival Predictive Markers at baseline
for FOLFIRI/FOLFOX vs FOLFIRI/FOLFOX + C225 KRAS Mutant
Below is a list of analytes that is predictive univariately for overall survival using the
Cox Proportional Hazard model with significant Treatment and Analyte interaction term.
On the right hand side, it indicates the median survival time and its 95% CI for the
FOLFIRI/FOLFOX arm and the FOLFIRI/FOLFOX + C225 arm using the specified cutoff.

| | Treatment × | | | | FOLFIRI/FOLFOX | | F/F + C225 | |
|---|---|---|---|---|---|---|---|---|
| | Analyte | | | | Median | | Median | |
| Analyte | p-value | Cutoff | HR | 95% CI | Survival | 95% CI | Survival | 95% CI |
| | Median | | | | | | | |
| egf | 0.0137 | <median | 3.03 | (1.13, 8.16) | 34.5 | (29.7, ∞) | 17.2 | (11.6, ∞) |
| | | 0.0282* | | | | | | |
| | Perm | | | | | | | |
| | 0.03 | | | | | | | |
| | continuous | | | | | | | |
| egf | 0.03 | | | | | | | |
| | Median | | | | | | | |
| erbb3 | 0.30 | >median | | | | | | |
| | | 0.81* | | | | | | |

*corresponds to the hazard

### REFERENCES

Cunningham et al., New England journal of medicine 351.4 (2004): 337-345.
Jonker et al., New England Journal of Medicine 357.20 (2007): 2040-2048.
Van Cutsem et al., New England Journal of Medicine 360.14 (2009): 1408-1417.
Van Cutsem et al., Journal of clinical oncology 25.13 (2007): 1658-1664.
Chung et al., Journal of Clinical Oncology 23.9 (2005): 1803-1810.
Hecht et al., Clinical Cancer Research 16.7 (2010): 2205-2213.
Laurent-Puig et al., Journal of Clinical Oncology 27.35 (2009): 5924-5930.
Moroni et al., The lancet oncology 6.5 (2005): 279-286.
Sartore-Bianchi et al., Journal of Clinical Oncology 25.22 (2007): 3238-3245.
Scartozzi et al., BMC cancer 9.1 (2009): 303.
Amado et al., European Journal of Cancer Supplements 5.6 (2007): 8.
De Roock et al., The lancet oncology 11.8 (2010): 753-762.
De Roock et al., Annals of Oncology 19.3 (2008): 508-515.
Di Fiore et al., British journal of cancer 96.8 (2007): 1166-1169.
Lievre et al., Cancer research 66.8 (2006): 3992-3995.
Bos et al., Nature 327.6120 (1987): 293-297.
Allegra et al., Journal of Clinical Oncology 27.12 (2009): 2091-2096.
Di Nicolantonio et al., Journal of Clinical Oncology 26.35 (2008): 5705-5712.
Erben et al., International Journal of Radiation Oncology*Biology*Physics 81.4 (2011): 1032-1038.
Laurent-Puig, et al., Clinical Cancer Research 15.4 (2009): 1133-1139.
Li, Xinqun, and Zhen Fan, Cancer research 70.14 (2010): 5942-5952.
Loupakis et al., British journal of cancer 101.4 (2009): 715-721.
Modest et al., International journal of cancer 131.4 (2012): 980-986.
Prenen et al., Clinical Cancer Research 15.9 (2009): 3184-3188.
Roth et al., Journal of Clinical Oncology 28.3 (2010): 466-474.
Sun et al., Gut (2012): gutjnl-2012.
Baker et al., British journal of cancer 104.3 (2011): 488-495.
Jacobs et al., Journal of clinical oncology 27.30 (2009): 5068-5074.
Khambata-Ford et al., Journal of Clinical Oncology 25.22 (2007): 3230-3237.
Venook et al., Journal of Clinical Oncology 24 (2006); ASCO Annual Meeting Proceedings. Vol. 24. No. 18 suppl. 2006.

1. A method of predicting responsiveness of a cancer in a subject to a cancer therapy including a EGFR targeting agent comprising: obtaining a biological sample from the subject; measuring an expression level of at least one biomarker selected from CD73, HER3, EGF, EGFR, HB-EGF, BTC, HER2, HER4, and DUSP4 in the sample from the subject; generating a comparison of the expression level of the biomarker in the sample to a reference level of the biomarker; and using said comparison to predict the responsiveness of the cancer to treatment with the cancer therapy including a EGFR targeting agent.

2. The method of claim 1, wherein the expression level of the biomarker is the protein expression level or the mRNA expression level.

3. The method of claim 2, wherein the biological sample is a blood sample, plasma sample, tumor sample or cancer cell sample.

4. The method of claim 3, wherein CD73 is measured and the prediction indicates responsiveness to a EGFR targeting agent when the protein expression level of CD73 is more than 4.3 ng/mL.

5. The method of claim 3, wherein HER3 is measured and the prediction indicates responsiveness to a EGFR targeting agent when the protein expression level of HER3 is more than 11 ng/mL.

6. (canceled)

7. (canceled)

8. (canceled)

9. (canceled)

10. (canceled)

11. The method of claim 3, wherein CD73 is measured and the prediction indicates responsiveness to a EGFR targeting agent when the mRNA expression level of CD73 is higher than a reference level.

12. The method of claim 3, wherein HER3 is measured and the subject is KRAS-WT, and wherein the prediction indicates lack of responsiveness to a EGFR targeting agent when the mRNA expression level of HER3 is higher than a reference level.

13. (canceled)

14. (canceled)

15. (canceled)

16. (canceled)

17. (canceled)

18. The method of claim 1, wherein the cancer is selected from the group consisting of colorectal, pancreatic, liver, esophageal, gastric, small bowel, cholangiocarcinoma, lung, head and neck, thyroid, melanoma, breast, renal, bladder, ovarian, uterine, prostate, lymphomas, leukemias, neuroendocrine, glioblastoma or any other form of brain cancer.

19. The method of claim 1, wherein the cancer is colorectal cancer.

20. The method of claim 1, further comprising administering an EGFR targeting agent to the subject if the cancer is predicted to be responsive to the EGFR targeting agent.

21. The method of claim 1, wherein the cancer therapy comprises a chemotherapy agent.

22. The method of claim 1, wherein the EGFR targeting agent is cetuximab.

23. A method of developing a prognosis for a subject diagnosed with cancer comprising: obtaining a biological sample from the subject; measuring an expression level of at least one biomarker selected from CD73, HER2, EREG, EGF, EGFR, HB-EFG, and HER3 in the sample from the subject; generating a comparison of the expression level of the biomarker in the sample to a reference level of the biomarker; and using said comparison to determine a survival prognosis for the subject.

24. The method of claim 23, wherein the expression level of the biomarker is the protein expression level or the mRNA expression level.

25. The method of claim 24, wherein the biological sample is a blood sample, serum sample, tumor sample or cancer cell sample.

26. The method of claim 25, wherein CD73 is measured and an expression level of CD73 less than 4.3 ng/mL is indicative of a better prognosis.

27. (canceled)

28. (canceled)

29. (canceled)

30. (canceled)

31. (canceled)

32. (canceled)

33. (canceled)

34. (canceled)

35. (canceled)

36. (canceled)

37. The method of claim 23, wherein the cancer is selected from the group consisting of colorectal, pancreatic, liver, esophageal, gastric, small bowel, cholangiocarcinoma, lung, head and neck, thyroid, melanoma, breast, renal, bladder, ovarian, uterine, prostate, lymphomas, leukemias, neuroendocrine, glioblastoma or any other form of brain cancer.

38. (canceled)

39. The method of claim 23, wherein a better prognosis indicates longer overall survival or longer progression free survival as compared to controls.

40. A method of treating cancer in a subject, comprising having determined an expression level of at least one biomarker selected from CD73, HER3, EGF, EGFR, HB-EGF, BTC, HER2, HER4, and DUSP4 in a biological sample from the subject; selecting a treatment regimen for the subject based on the expression of at least one of the biomarkers, and administering a therapeutically effective amount of an EGFR targeting agent in the subject if the cancer is predicted to be responsive to the EGFR targeting agent.

41. The method of claim 40, wherein the expression level of the biomarker is the protein expression level, or the mRNA expression level.

42. The method of claim 41, wherein the biological sample is a blood sample, serum sample, tumor sample or a cancer cell sample.

43. The method of claim 42, wherein the biomarker comprises CD73 and the treatment regimen comprises a EGFR targeting agent when the protein expression level of CD73 is more than 4.3 ng/mL.

44. The method of claim 42, wherein the biomarker comprises HER3 and the treatment regimen comprises a EGFR targeting agent when the protein expression level of HER3 is more than 11 ng/mL.

45. (canceled)

46. (canceled)

47. (canceled)

48. (canceled)

49. (canceled)

50. The method of claim 42, wherein the biomarker comprises CD73 and the treatment regimen comprises a EGFR targeting agent when the mRNA expression level of CD73 is higher than a reference level.

51. The method of claim 42, wherein the biomarker comprises HER3 and the subject is KRAS-WT and the treatment regimen comprises a EGFR targeting agent when the mRNA expression level of HER3 lower than a reference level.

52. (canceled)

53. (canceled)

54. (canceled)

55. (canceled)

56. (canceled)

57. The method of claim 40, wherein the cancer is selected from the group consisting of colorectal, pancreatic, liver, esophageal, gastric, small bowel, cholangiocarcinoma, lung, head and neck, thyroid, melanoma, breast, renal, bladder, ovarian, uterine, prostate, lymphomas, leukemias, neuroendocrine, glioblastoma or any other form of brain cancer.

58. (canceled)

59. The method of claim 40, wherein the treatment regimen further comprises a chemotherapy agent.

60. The method of claim 40, wherein the EGFR targeting agent is cetuximab.
